(19) United States
(12) Patent Application Publication
(10) Pub. No.: US 2025/0269138 A1
(43) Pub. Date: Aug. 28, 2025

(54) VENT AND PRESSURE REGULATING DEVICE

(71) Applicant: Fisher & Paykel Healthcare Limited, Auckland (NZ)

(72) Inventors: Zak Jake FLINTOFF, Auckland (NZ); Frederick David Allan WRIGHT, Auckland (NZ); Madison Brooke CHU, Auckland (NZ)

(21) Appl. No.: 18/847,154

(22) PCT Filed: Mar. 14, 2023
(86) PCT No.: PCT/IB2023/052432

§ 371 Date: Sep. 13, 2024

Related U.S. Application Data
(60) Provisional application No. 63/369,020, filed on Jul. 21, 2022.
(60) Provisional application No. 63/269,289, filed on Mar. 14, 2022.

(51) Int. Cl. A61M 16/20; A61M 16/00; A61M 16/08

(52) U.S. Cl. CPC A61M 16/201; A61M 16/0003; A61M 16/0833; A61M 2016/0027; A61M 2016/003

(57) ABSTRACT

A vent for use with a respiratory system arranged to convey a breathable gas to a patient, wherein the vent allows gas from within the respiratory system to exit, comprising: a movable actuator, wherein movement of the actuator adjusts an area of the vent available for the gas to exit from the respiratory system via the vent.

### TECHNICAL FIELD

The present disclosure relates to various devices, systems, and methods applicable to a respiratory system arranged to deliver a breathable gas to a patient. In at least one aspect, the present disclosure relates to a vent for use with a respiratory system. In at least another aspect, the pressure disclosure relates to a pressure regulating device including a vent and a flow independent PEEP valve.

### CROSS REFERENCE

The present application is related to U.S. provisional application 63/269,289, filed on 14 Mar. 2022, U.S. provisional application 63/369,020, filed on 21 Jul. 2022 and U.S. provisional application 63/366,660, filed on 20 Jun. 2022, the contents of which are incorporated herein by reference in their entirety.

### BACKGROUND

Positive End Expiratory Pressure (PEEP) and/or Peak Inspiratory Pressure (PIP) can be controllably provided to a patient during respiration, resuscitation or assisted respiration (ventilation).

PEEP is a pressure delivered to the patient throughout the expiratory phase of positive pressure ventilation, resuscitation, or assisted respiration. PIP is a desired highest pressure provided to the patient during the inspiratory phase of positive pressure ventilation, resuscitation, or assisted respiration. The patients may be neonates or infants who require breathing assistance or resuscitation. In applying PEEP or PIP, the patient's upper airway and lungs are held open by the applied pressure. During resuscitation, fluid filled in the lungs is displaced and air takes its place. This is a delicate process as over-inflation of the lungs poses the risk of IVH (Intraventricular haemorrhage) and lung damage.

Self-inflating or flow-inflating bags can be used to provide respiratory support to a patient. Self-inflating bags may deliver pressure in ‘spikes’ or ‘pulses’ (see for example ‘pressure over time’ waveform 5003 in FIG. 6), with no or limited PEEP, overly high PIP and/or relatively short inspiratory time, which means there is a sudden rise and fall in pressure delivered. This may pose the risk of potential lung damage to the patient. Compared to self-inflating and flow-inflating bags, T-piece resuscitators deliver a more controlled and consistent PIP and PEEP (see for example ‘pressure over time’ 5001 in FIG. 6), which helps to protect the newborn's lungs.

Any reference to or discussion of any document, act or item of knowledge in this specification is included solely for the purpose of providing a context for the present invention. It is not suggested or represented that any of these matters or any combination thereof formed at the priority date part of the common general knowledge, or was known to be relevant to an attempt to solve any problem with which this specification is concerned.

### SUMMARY OF THE INVENTION

In a first aspect, the present disclosure provides a vent for use with a respiratory system arranged to convey a breathable gas to a patient, wherein the vent allows gas from within the respiratory system to exit, comprising:
a movable actuator, wherein movement of the actuator adjusts an area of the vent available for the gas to exit from the respiratory system via the vent.

In some embodiments, adjusting the area of the vent available for the gas to exit regulates a pressure of the breathable gas delivered to the patient.

In some embodiments, the vent includes an orifice, wherein the gas is arranged to flow through the orifice when exiting from the respiratory system.

In some embodiments, the orifice is gradually occluded as the actuator is moved in a first direction. The occlusion of the orifice results in an increase of the pressure of the breathable gas delivered to the patient.

In some embodiments, the orifice is gradually unoccluded as the actuator is moved in a second direction. The unocclusion of the orifice results in a decrease of the pressure of the breathable gas delivered to the patient.

In some embodiments, when the orifice is fully or substantially occluded, the pressure of the breathable gas delivered to the patient is higher than the pressure of the breathable gas delivered to the patient when the orifice is fully or substantially unoccluded.

In some embodiments, when the orifice is fully or substantially occluded, the pressure of the breathable gas delivered to the patient corresponds to PIP, or peak inspiratory pressure. When the orifice is fully or substantially unoccluded, the pressure of the breathable gas delivered to the patient corresponds to PEEP, or positive end expiratory pressure. When the orifice is partially occluded or unoccluded, pressures between PEEP and PIP are delivered to the patient.

In some embodiments, a shape or configuration of the orifice is configured based at least in part on a desired rate of occlusion or unocclusion of the orifice during movement of the actuator.

In some embodiments, the shape of the orifice is configured based at least in part on inspiration to expiration ratio (I:E) supplied to the patient.

In some embodiments, the shape of the orifice is configured based on a desired pressure waveform of the breathable gas to be delivered to the patient.

In some embodiments, the shape of the orifice is configured such that a rate of occlusion or unocclusion by the actuator varies along a movement direction of the actuator, when the actuator is moved at a substantially constant speed.

In some embodiments, the pressure of the breathable gas is regulated in a substantially non-linear manner as the actuator is moved at a constant speed.

In some embodiments, the rate of occlusion or unocclusion of the orifice by the actuator is greater at one end of the orifice, such that a resulted change in the pressure of the breathable gas delivered to the patient varies more rapidly.

In some embodiments, the rate of occlusion or unocclusion by the actuator is smaller at another end of the orifice, such that the resulted change in the pressure of the breathable gas delivered to the patient varies less rapidly.

In some embodiments, the orifice is of a substantially circular shape.

In some embodiments, the orifice is of an oval shape.

In some embodiments, the orifice is of a triangular shape.

In some embodiments, the shape of the orifice is configured such that a rate of occlusion or unocclusion by the actuator remains substantially constant along a movement direction of the actuator, when the actuator is moved at a constant speed.

In some embodiments, the orifice is of a square, or a rectangular shape.

In some embodiments, the orifice is of an irregular shape, or a combination of the above-mentioned shapes.

In some embodiments, the movement speed of the actuator is at least partly manually controlled by the operator.

In some embodiments, the vent includes a support structure, wherein the actuator is movably coupled to and supported by the support structure.

In some embodiments, the actuator is slidably coupled to the support structure, and the area of the vent available for the gas to exit from the respiratory system is adjusted by sliding the actuator along the support structure.

In some embodiments, the support structure includes one or more channels, for guiding the movement of the actuator.

In some embodiments, the one or more channels include at least two substantially parallel channels.

In some embodiments, the one or more channels are formed along a peripheral portion of the support structure. In other embodiments, the one or more channels may be disposed on a surface of the support structure.

In some embodiments, the support structure includes a hinge, for guiding the movement of the actuator.

In some embodiments, the actuator is rotatable with respect to the hinge.

In some embodiments, the actuator includes an engaging member arranged to be engaged by a finger or digit of the operator when sliding the actuator.

In some embodiments, the engaging member includes a raised formation for example, a protrusion, button, ridge, and similar thereof, and/or an indented formation for example, a recess, groove, and similar thereof.

In some embodiments, the actuator and the support structure are made from one or more of plastic, foam, rubber materials, depending on friction of movement, comfort, sealing ability.

In some embodiments, the vent includes a vent cap.

In some embodiments, the vent cap includes one or more orifices, wherein the gas is arranged to exit from respiratory system via the one or more orifices.

In some embodiments, the one or more orifices are gradually occluded as the actuator is moved in a first direction, the occlusion of the one or more orifices results in an increase of the pressure of the breathable gas delivered to the patient.

In some embodiments, the one or more orifices are gradually unoccluded as the actuator is moved in a second direction, the unocclusion of the one or more orifices results in a decrease of the pressure of the breathable gas delivered to the patient.

In some embodiments, when the one or more orifices are fully or substantially occluded, the pressure of the breathable gas delivered to the patient is higher than the pressure of the breathable gas delivered to the patient when the one or more orifices are fully or substantially unoccluded.

In some embodiments, when the one or more orifices are fully or substantially occluded, the pressure of the breathable gas delivered to the patient corresponds to PIP, or peak inspiratory pressure. When the one or more orifices are fully or substantially unoccluded, the pressure of the breathable gas delivered to the patient corresponds to PEEP, or positive end expiratory pressure. When the one or more orifices are partially occluded or unoccluded, pressures between PEEP and PIP are delivered to the patient.

In some embodiments, the one or more orifices include an array of cut outs formed in the vent cap.

In some embodiments, the array of cut outs are formed as concentric circular cut-outs.

In some embodiments, the array of cut outs are formed as concentric elliptical cut-outs.

In some embodiments, the actuator includes a deformable portion arranged to occlude or unocclude the one or more orifices.

In some embodiments, the deformable portion elastically deforms when a force is applied to it, and returns to its shape when the force is removed.

In some embodiments, the actuator is pressed against the vent cap, such that the deformable portion gradually deforms, to occlude the one or more orifices.

In some embodiments, the deformable portion is configured such that an orifice, or a portion of an orifice, closer to a centre of the vent cap is occluded first.

In some embodiments, the deformable portion is formed in a dome shape.

In some embodiments, the deformable portion is at least partially formed from an elastic material.

In some embodiments, the elastic material includes silicone.

In some embodiments, the vent includes a support structure, for holding the actuator in close vicinity of the vent cap.

In some embodiments, the support structure includes a body, forming a cavity, wherein the vent cap is positioned inside the cavity.

In some embodiments, the support structures includes two or more elongate members extending upwardly from the body.

In some embodiments, the elongate members each includes a shoulder portion which extend inwardly toward the centre of the vent cap, wherein the shoulder portion of the elongate members assists to hold the actuator in place.

In some embodiment, the support structure includes a circular ring connecting the shoulder portions of the elongate members, wherein the circular ring and the shoulder portion of the elongate members assist to hold the actuator in place.

In some embodiments, the actuator is formed in a dome shape.

In some embodiments, the actuator is formed as a piston, and the deformable portion is located at a lower end of the piston.

In some embodiments, the deformable portion includes a hollow interior, to allow easier deforming when the actuator is pressed against the vent cap.

In some embodiments, a relief hole is provided in the deformable portion to reduce the resistance when pressing the actuator against the vent cap.

In some embodiments, the vent includes a housing, comprising:
a first and a second opening, wherein the first opening is fluidly connected to the respiratory system, and the second opening is configured to movably receive the actuator, wherein a position of the actuator with respect to the housing determines the area of the vent available for the gas to exit through the vent from the respiratory system, thereby adjusting the pressure of the breathable gas delivered to the patient.

In some embodiments, the actuator is arranged to be slid in or out of the housing via the second opening.

In some embodiments, the first opening is located at or near a lower end of the housing, and the second opening is located at near an upper end of the housing.

In some embodiments, the housing is of a substantially cylindrical shape.

In some embodiments, the actuator includes a hollow body, comprising:
one or more air inlets for receiving the gas from the respiratory system, and
a plurality of orifices arranged to allow the gas to exit from the hollow body.

In some embodiments, the hollow body is of a substantially cylindrical shape.

In some embodiments, the hollow body includes an upper end and lower end, and a side wall extending between the upper end and the lower end.

In some embodiments, the one or more air inlets are provided at or near the lower end of the hollow body.

In some embodiments, the plurality of orifices are formed in the side wall of the hollow body.

In some embodiments, the actuator is arranged to move between a first and a second position to control the pressure of the breathable gas delivered to the patient,
wherein in the first position the actuator is lifted with respect to the housing, such that the plurality of orifices are exposed to ambient air, and the gas can exit the vent via these orifices;
wherein in the second position the actuator is at least partially inserted into the housing, such that the plurality of orifices are not exposed to ambient air, and
wherein between the first and second position, one or more of the plurality of orifices are exposed to the atmosphere allowing the gas to exit through the vent.

In some embodiments, the plurality of orifices are configured to have varying shapes and/or sizes.

In some embodiments, the plurality of orifices are disposed along the side wall of the hollow body, and a first orifice formed near the open end of the actuator has a larger size compared to remaining orifices.

In some embodiments, the actuator is formed as an elongate plunger.

In another embodiment, the actuator includes:
a body portion, including a first end and a tapering end, wherein a diameter of the body portion decreases towards the tapering end.

In some embodiments, the actuator is arranged to move between a first and a second position to control the pressure of the breathable gas delivered to the patient,
wherein in the first position the actuator is lifted with respect to the housing, such that the gas is allowed to flow via a gap between the actuator and an internal wall of the housing, and
wherein in the second position the actuator is at least partially inserted into the housing, such that the gap between the actuator and the internal wall of the housing reduces in size, and/or is substantially blocked, such that the gas does not flow through the gap.

In some embodiments, when the actuator is in the second position, the pressure of the breathable gas delivered to the patient is higher than the pressure of the breathable gas delivered to the patient when the actuator is in the first position.

In some embodiments, when the actuator is in the first position, the pressure of the breathable gas delivered to the patient corresponds to PEEP. When the actuator is in the second position, the pressure of the breathable gas delivered to the patient corresponds to PIP. As the actuator is moved between the first and second positions, pressures between PEEP and PIP are delivered to the patient.

In some embodiments, the actuator is manually operated by an operator to move between the first position and the second position.

In some embodiments, the actuator is pressed downwards towards the housing by the operator, to move from the first position to the second position.

In some embodiments, the actuator is allowed to gradually return to the first position, as the operator reduces or removes the force applied to the actuator. In other embodiments, the actuator may be pulled in an upward direction by the operator, to move from the second position to the first position.

In some embodiments, the vent further includes a biasing member to cause the actuator to remain in the first position when no force is applied by the operator.

In some embodiments, the actuator includes a shoulder disposed on an exterior surface of the actuator.

In some embodiments, the housing includes a recess, wherein the biasing member is held in place by the shoulder of the actuator and the recess of the housing.

In some embodiments, the housing includes a countersunk hollow, wherein the biasing member is held in place by the shoulder of the actuator and the countersunk hollow of the housing.

In some embodiments, the shoulder is formed as a flange which partially or fully extends around a circumference of the actuator.

In some embodiments, the biasing member is a spring disposed on the outside of the actuator.

In some embodiments, a sealing member is provided to create a seal when the actuator is in the first position.

In some embodiments, the sealing member is an O-ring.

In some embodiments, the vent includes a housing, comprising:
a first and a second opening, wherein the first opening is configured to fluidly connect to the respiratory system, and the second opening is configured to movably receive the actuator within the housing;
a body extending between the first and the second opening, wherein the body includes one or more orifices configured to allow the gas to escape to atmosphere depending on a relative position of the actuator with respect to the housing.

In some embodiments, the actuator is arranged to move between a first and a second position to adjust the pressure of the breathable gas delivered to the patient,
wherein in the first position the actuator is lifted with respect to the housing, such that the gas is allowed to flow through the vent via the one or more orifices and exit from the respiratory system, and
wherein in the second position the actuator is lowered into the housing, to block the first opening of the housing.

In some embodiments, when the actuator is in the second position, the pressure of the breathable gas delivered to the patient is higher than the pressure delivered when the actuator is in the first position.

In some embodiments, when the actuator is in the first position, the pressure of the breathable gas delivered to the patient corresponds to PEEP, and when the actuator is in the second position, the pressure of the breathable gas delivered to the patient corresponds to PIP. As the actuator is moved between the first and second positions, pressures between PEEP and PIP are delivered to the patient.

In some embodiments, the one or more orifices include a plurality of orifices which are formed in the body of the housing.

In some embodiments, the plurality of orifices are disposed around a circumference of the body and extend along a length of the body.

In some embodiments, the vent includes a deformable membrane which assists with movement of the actuator.

In some embodiments, the membrane forms a chamber extending between the second opening of the housing and a shoulder of the actuator.

In some embodiments, the membrane is configured such that it biases the actuator in the first position when no force is applied to the actuator.

In some embodiments, the membrane is configured such that as a pressing force is applied to the actuator, it deforms to allow the actuator to move towards the second position.

In some embodiments, the membrane is configured such that a portion of the membrane moves the actuator into the second position, as the actuator moves past a deflection point of the membrane.

In some embodiments, the membrane is configured such that a portion of the membrane deflects and moves the actuator into the second position, as the actuator moves past a deflection point of the membrane.

In some embodiments, the membrane returns the actuator back to the first position as the pressing force is removed from the actuator.

In some embodiments, the actuator includes a substantially cylindrical body, wherein a bottom surface of the cylindrical body may have a curved or a substantially flat surface.

In some embodiments, the actuator includes a sealing portion disposed on the body of the actuator.

In some embodiments, the sealing portion is a protrusion raised above a surface of the body of the actuator, which extends partially or substantially around a circumference of the body.

In some embodiments, the protrusion includes an angled surface.

In some embodiments, the sealing portion of the actuator is configured to engage a complimentary sealing portion provided in the vent housing.

In some embodiments, the complimentary sealing portion includes a recess, or a chamfer, formed in the body of the vent housing.

In some embodiments, the complimentary sealing portion includes an angled surface, configured to engage the sealing portion of the actuator when the actuator is in the second position.

In some embodiments, the vent includes a member which assists with controlling the speed of movement of the actuator.

In some embodiments, the member is configured to contact the actuator during its movement, to apply a friction force onto the actuator.

In some embodiments, the friction force is greater when the actuator is moved from the second position to the first position.

In some embodiments, the member comprises one or more flaps which are arranged to deform, and/or deflect during movement of the actuator.

In some embodiments, the vent is permanently attached to the respiratory system.

In some embodiments, the vent includes connector portions allowing it to be detachably connected to the respiratory system.

In some embodiments, the connector portions include threaded portions, configured to allow the vent to be threaded to complimentary connector portions provided in the respiratory system.

In some embodiments, the vent is used in conjunction with a pressure regulating valve.

In some embodiments, the valve comprises:
a valve body including an inlet and an outlet, said inlet configured to be in fluid communication with the respiratory system, said outlet configured to be in fluid communication with the vent;
a controller disposed within the valve body, in a flow path between the valve inlet and the outlet,
wherein the controller is movable by the gas, movement of the controller being at least partially dependent on a pressure of the gas at the inlet, wherein said movement adjusts the flow path between the inlet and the outlet of the valve, to regulate the pressure of the gas in the respiratory system within a predetermined range.

In some embodiments, the controller is biased towards the valve inlet.

In some embodiments, the valve comprises:
a valve body including an inlet and an outlet, said inlet configured to be in fluid communication with the respiratory system;
a controller disposed within the valve body, in a flow path between the inlet and the outlet,
wherein the controller is biased towards the inlet and movement of the controller away from the inlet being at least partially dependent on a pressure of the gas at the inlet, said movement adjusting the flow path between the inlet and the outlet, to regulate the pressure of the gas in the respiratory system within a predetermined range.

In some embodiments, the controller is caused to move away from the inlet when the pressure exceeds a selected pressure level.

In some embodiments, the predetermined range is a predetermined PEEP pressure range.

In some embodiments, the selected pressure level is within the predetermined PEEP pressure range.

In some embodiments, the valve comprises:
a valve body including an inlet and an outlet, said inlet configured to be in fluid communication with the respiratory system;
a controller disposed within the valve body, in a flow path between the inlet and the outlet,
wherein the controller is biased towards the inlet, the controller being caused to move away from the inlet when the pressure exceeds a selected pressure level, said movement adjusting the flow path between the inlet and the outlet, to regulate the pressure of the gas in the respiratory system within a predetermined PEEP pressure range, wherein the selected pressure level is within the predetermined PEEP pressure range.

In some embodiments, the movement of the controller is at least partially dependent on a pressure differential between an upper and a lower surface of the controller.

In some embodiments, the valve comprises a biasing member, operatively coupled to the controller.

In some embodiments, the controller is biased towards the inlet by the biasing member.

In some embodiments, the biasing member applies a variable resistance force onto the controller during movement thereof.

In some embodiments, the variable resistance force applied by the biasing member counters a force exerted onto the controller caused by the pressure differential between the upper and lower surfaces of the controller.

In some embodiments, the flow path between the inlet and the outlet of the valve is closed off, when the pressure differential is below the selected pressure level.

In some embodiments, when the pressure differential is above the selected pressure level, it overcomes the variable resistance force applied onto the actuator by the biasing member, and opens up the flow path between the inlet and the outlet of the valve.

In some embodiments, the biasing member biases the controller against a valve seat when the flow path is closed off.

In some embodiments, the flow path between the inlet and the outlet of the valve is opened up when the controller is displaced from the valve seat.

In some embodiments, the flow path is at least in part determined by a relative displacement of the controller from the valve seat.

In some embodiments, the valve includes a supporting member for the controller, for guiding and stabilising the movement of the controller.

In some embodiments, the supporting member is an elongate shaft, along which the controller is arranged to slide during its movement.

In some embodiments, the valve outlet is fluidly connected to the first opening of the vent.

In some embodiments, the pressure of the gas within the respiratory system is at least in part determined by flow variations caused by unintentional leaks, such as interface leak, and/or auto-PEEP, which causes the pressure to be lower or higher than a targeted PEEP pressure to be delivered to the patient.

In some embodiments, the valve is arranged to compensate for the unintentional leaks, by reducing the gas flow through the valve.

In some embodiments, the valve is arranged to compensate for auto-PEEP, by allowing a variable portion of the gas within the respiratory system to flow through the valve while substantially maintaining pressure delivered to the patient.

In some embodiments, the predetermined PEEP pressure range is between 5 and 12 cm H₂O.

In some embodiments, the movement of the controller is able to regulate the pressure of the breathable gas within the respiratory system by a variation of −2 to +2 cm H₂O, −1 to +1 cm H₂O, or 0.5 to +0.5 cm H₂O.

In some embodiments, the selected pressure level is within a range of 4.5 to 5.5 cm H₂O, or 4 to 6 cm H₂O, or 3 to 7 cm H₂O.

In some embodiments, the valve is a PEEP valve.

In a second aspect, the present disclosure provides a pressure regulating device for use with a respiratory system arranged to convey a breathable gas to a patient, wherein the pressure regulating device allows gas from within the respiratory system to exit, comprising:
a vent according to the first aspect of the present disclosure;
a valve operatively coupled to the vent, for regulating a pressure of the breathable gas within the respiratory system within a predetermined pressure range.

In some embodiments, the valve comprises:
a valve body including an inlet and an outlet, said inlet configured to be in fluid communication with the respiratory system;
a controller disposed within the valve body, in a flow path between the valve inlet and the outlet,
wherein the controller is movable by the gas, movement of the controller being at least partially dependent on a pressure of the gas at the inlet, wherein said movement adjusts the flow path between the inlet and the outlet of the valve, to regulate the pressure of the gas in the respiratory system within the predetermined range.

In some embodiments, the movement of the controller is at least partially dependent on a pressure differential between an upper and a lower surface of the controller.

In some embodiments, the controller is biased towards the valve inlet, by a biasing member.

In some embodiments, the controller is caused to move away from the inlet when the pressure differential exceeds a selected pressure level.

In some embodiments, the biasing member applies a variable resistance force onto the controller during movement thereof.

In some embodiments, the variable resistance force applied by the biasing member counters a force exerted onto the controller caused by the gas pressure at the valve inlet.

In some embodiments, the flow path between the inlet and the outlet of the valve is closed off, when the pressure differential is below the selected pressure level.

In some embodiments, when the pressure differential is above the selected pressure level, it overcomes the variable resistance force applied onto the actuator by the biasing member, and opens up the flow path between the inlet and the outlet of the valve.

In some embodiments, the biasing member biases the controller against a valve seat.

In some embodiments, the flow path between the inlet and the outlet of the valve is opened up when the controller is displaced from the valve seat.

In some embodiments, the valve includes a supporting member for the controller, for guiding and stabilising the movement of the controller.

In some embodiments, the supporting member is an elongate shaft, along which the controller is arranged to slide during its movement.

In some embodiments, the valve outlet is fluidly connected to the first opening of the vent.

In some embodiments, the biasing member is configured to be retained in a compressed state within the valve body, when the actuator is biased against the valve seat.

In some embodiments, the pressure of the gas within the respiratory system is at least in part determined by flow variations caused by unintentional leaks and/or auto-PEEP, which causes the pressure to be lower or higher than a targeted PEEP pressure to be delivered to the patient.

In some embodiments, the valve is arranged to compensate for the unintentional leaks, by reducing the gas flow through the valve.

In some embodiments, the valve is arranged to compensate for auto-PEEP, by allowing a variable portion of the gas within the respiratory system to flow through the valve substantially maintaining pressure delivered to the patient.

In some embodiments, the predetermined range is a predetermined PEEP pressure range.

In some embodiments, the selected pressure level is within the predetermined PEEP pressure range.

In some embodiments, the movement of the controller is able to regulate the pressure of the breathable gas within the respiratory system by a variation of −2 to +2 cm H₂O, −1 to +1 cm H₂O, or −0.5 to +0.5 cm H₂O.

In some embodiments, the selected pressure level is within a range of 4.5 to 5.5 cm H₂O, or 4 to 6 cm H₂O, or 3 to 7 cm H₂O.

In some embodiments, the pressure regulating device is configured to be removably attachable to a venting orifice of the respiratory system.

In some embodiments, the venting orifice is provided in a T-piece device.

In a third aspect, the present disclosure provides a device for use with a respiratory system, wherein the device comprises:
a housing, including:
   an inlet arranged to receive a breathable gas from a respiratory apparatus;
   an outlet configured to be in fluid communication with an airway of the patient;
   a PEEP port, wherein the PEEP port is configured to fluidly connect to a vent or a pressure regulating device according to the first or second aspect of this disclosure.

In some embodiments, the device is a T-piece device.

In some embodiments, the device additionally includes an optional opening for insertion of one or more auxiliary equipment including one or more of a catheter for fluid clearance or surfactant delivery to the patient and/or a monitoring device for monitoring one or more parameters of inhaled and/or exhaled gas.

In some embodiments, the outlet of the device is fluidly connectable to, or arranged to be in fluid communication with a patient interface.

In some embodiments, the respiratory apparatus is a resuscitation device or includes a flow generator.

In a fourth aspect, the present disclosure provides a kit of parts for use with a respiratory system, the kit of parts comprising:
a vent according to the first aspect or a pressure regulating device according to the second aspect of the present disclosure; and
a T-piece device, wherein the vent or the pressure regulating device is connectable to a PEEP port of the T-piece device.

In some embodiments, the kit of parts includes a patient interface, connectable to a port of the T-piece device.

In some embodiments, the patient interface includes a range of different types, sizes and/or fit. The patient interface may include a suitable interface, such as a mask, including nasal or full-face mask, nasal cannula or endotracheal (ET) tube. The patient interface may be an interface capable of creating a seal with at least one patient airway.

In some embodiments, the kit of parts includes a flexible hose, connectable to an inlet of the T-piece device.

In some embodiments, the kit of parts includes one or more conduits, connectable to a respiratory apparatus to receive a flow of breathable gas therefrom.

In some embodiments, the kit of parts includes connectors, for establishing connections between the vent and the T-piece device, and/or between the pressure regulating device and the T-piece device, and/or between the one or more conduits and the respiratory apparatus, and/or between the T-piece device and the flexible hose.

In a fifth aspect, the present disclosure provides a respiratory system for delivering a respiratory therapy to a patient, the respiratory system comprising:
a respiratory apparatus, which supplies a source of breathable gas flow at a targeted pressure and/or flow rate;
a conduit assembly connectable to the respiratory apparatus to receive the breathable gas flow;
a patient interface, arranged to receive the breathable gas and usable to deliver the respiratory therapy to the patient;
a device arranged to form a fluid connection between the conduit assembly and the patient interface; and
a vent according to the first aspect of the present disclosure, or a pressure regulating device according to the second aspect of the present disclosure.

In some embodiments, the respiratory system is connectable to a gas source, which can be a wall mounted gas supply.

In some embodiments, the respiratory system may additionally include a humidifier, for humidifying the breathable gas before it is conveyed to the patient.

In some embodiments, the device includes a housing, including:
an inlet arranged to receive the breathable gas from the respiratory apparatus;
an outlet configured to be in fluid communication with an inlet of the patient interface;
a PEEP port arranged to allow gas from within the respiratory system to exit from the respiratory system to ambient air.

In some embodiments, the vent or the pressure regulating device is connectable to the PEEP port of the device.

In a sixth aspect, the present disclosure provides a vent for use with a respiratory system arranged to convey a breathable gas to a patient, wherein the vent allows gas from within the respiratory system to exit, the vent comprising: a movable actuator configured to cover or uncover a port of the vent, wherein the port, when not covered, allows the gas from within the respiratory system to exit; and a member which assists with controlling the speed of movement of the actuator.

In some embodiments, the member is configured to contact the actuator during its movement, to apply a friction force onto the actuator. In some embodiments, the friction force is greater when the actuator is moved from a position where the an area of the vent available for the gas to exit from the respiratory system via the vent is minimum to another position where the an area of the vent available for the gas to exit from the respiratory system via the vent is maximum.

In some embodiments, the member comprises one or more flaps which are arranged to deform, and/or deflect during movement of the actuator.

In a seventh aspect, the present disclosure provides a device for use with a respiratory system, wherein the device comprises: a housing, including: an inlet arranged to receive a breathable gas from a respiratory apparatus; an outlet configured to be in fluid communication with an airway of the patient; a PEEP port, wherein the PEEP port is configured to fluidly couple to a vent, the vent comprising: a movable actuator, wherein movement of the actuator adjusts an area of the vent available for the gas to exit from the respiratory system via the pressure regulating device.

In an eighth aspect, the present disclosure provides a pressure regulating device for use with a respiratory system arranged to convey a breathable gas to a patient, wherein the pressure regulating device allows gas from within the respiratory system to exit and is configured to regulate a pressure of the breathable gas within the respiratory system within a predetermined pressure range, comprising: a vent, wherein the vent comprises: a movable actuator, wherein movement of the actuator adjusts an area of the vent available for the gas to exit from the respiratory system via the pressure regulating device.

In a ninth aspect, the present disclosure provides a respiratory system for delivering a respiratory therapy to a patient, the respiratory system comprising: a respiratory apparatus, which supplies a source of breathable gas flow at a targeted pressure and/or flow rate; a conduit assembly connectable to the respiratory apparatus to receive the breathable gas flow; a patient interface, arranged to receive the breathable gas and usable to deliver the respiratory therapy to the patient; a device arranged to form a fluid connection between the conduit assembly and the patient interface; and a vent or a pressure regulating device including the vent, wherein the vent comprises a movable actuator, and the pressure regulating device is configured to regulate a pressure of the breathable gas within the respiratory system within a predetermined pressure range, and wherein movement of the actuator adjusts an area of the vent available for the gas to exit from the respiratory system via the vent.

In a tenth aspect, the present disclosure provides a kit of parts for use with a respiratory system, the kit of parts comprising: a vent or a pressure regulating device including the vent, wherein the vent comprises a movable actuator, and the pressure regulating device is configured to regulate a pressure of the breathable gas within the respiratory system within a predetermined pressure range, and wherein movement of the actuator adjusts an area of the vent available for the gas to exit from the respiratory system via the vent; and a T-piece device, wherein the vent or the pressure regulating device is connectable to a PEEP port of the T-piece device.

In an eleventh aspect, the present disclosure provides a Continuous Positive Airway Pressure (CPAP) system, comprising: a respiratory apparatus, which supplies a source of breathable gas flow at a targeted pressure and/or flow rate; a conduit assembly including: an inspiratory breathing conduit connectable to the respiratory apparatus to receive the breathable gas flow; and an expiratory breathing conduit; a patient interface, arranged to receive the breathable gas and usable to deliver the respiratory therapy to the patient; a device arranged to form a fluid connection between the inspiratory breathing conduit and the patient interface; a vent or a pressure regulating device including the vent, wherein the vent comprises a movable actuator, and the pressure regulating device is configured to regulate a pressure of the breathable gas within the respiratory system within a predetermined pressure range, and wherein movement of the actuator adjusts an area of the vent available for the gas to exit from the respiratory system via the vent; and one or more connector portions configured to detachably connect the vent or the pressure regulating device with the expiratory breathing conduit.

Further features and advantages of the present disclosure will become apparent from the following detailed description.

### BRIEF DESCRIPTION OF THE DRAWINGS

Various preferred embodiments of the present disclosure will now be described, by way of examples only, with reference to the accompanying figures, in which:

FIG. 1 illustrates an exemplary respiratory system according to the present disclosure;

FIG. 2 shows another exemplary respiratory system according to the present disclosure;

FIG. 3A shows an exemplary T-piece device with an occluded PEEP port according to the present disclosure;

FIG. 3B shows the exemplary T-piece device of FIG. 3A with an unoccluded PEEP port according to the present disclosure;

FIG. 4A shows another exemplary T-piece device with an occluded PEEP port according to the present disclosure;

FIG. 4B shows the exemplary T-piece device of FIG. 4A with an unoccluded PEEP port according to the present disclosure;

FIG. 5 illustrates directions of gas flows in an example of a T-piece device;

FIG. 6 shows a comparison of pressure waveforms generated by a T-piece resuscitator and a self-inflating bag;

FIG. 7A shows a see-through perspective view of an embodiment of a vent connected to a device of the respiratory system;

FIG. 7B shows a see-through side view of the vent of FIG. 7A;

FIG. 8A shows an embodiment of a vent, with its actuator in a first position;

FIG. 8B shows the vent of FIG. 8A, with its actuator in a second position;

FIGS. 9A, 9B and 9C show various exemplary configurations of a vent, each having a different shaped orifice;

FIG. 10 illustrates how an exemplary circular orifice of a vent may be gradually opened or closed;

FIG. 11 is a diagram showing how an area of the circular orifice available for gas to flow through may vary over time;

FIG. 12 is a diagram showing how a pressure of a breathable gas delivered to a patient may vary with time, corresponding to the area of the circular orifice available for gas to flow through over time of FIG. 11;

FIG. 13 illustrates how an exemplary triangular orifice of a vent may be gradually closed;

FIG. 14 is a diagram showing an example of how an area of the triangular orifice available for gas to flow through may vary over time;

FIG. 15 is a diagram showing how a pressure of a breathable gas delivered to a patient may vary with time, corresponding to the area of the triangular orifice available to flow through over time of FIG. 14;

FIG. 16 illustrates another example of how a triangular orifice of a vent may be gradually opened;

FIG. 17 is a diagram showing another example of how an area of the triangular orifice available for gas to flow through may vary over time;

FIG. 18 is a diagram showing another example how a pressure of a breathable gas delivered to a patient may vary with time, corresponding to the area of the triangular orifice available to flow through over time of FIG. 17;

FIG. 19 illustrates how an exemplary rectangular orifice of a vent may be gradually closed;

FIG. 20 is a diagram showing an example of how an area of the rectangular orifice available for gas to flow through may vary over time;

FIG. 21 is a diagram showing an example of how a pressure of a breathable gas delivered to a patient may vary with time, corresponding to the area of the rectangular orifice available to flow through over time of FIG. 20;

FIG. 22 shows various exemplary configurations of an actuator of a vent;

FIG. 23 shows another exemplary vent, with its actuator in a range of different exemplary positions;

FIG. 24 shows a perspective view of the vent of FIG. 23, with its actuator fully covering an orifice of the vent;

FIG. 25 shows another perspective view of the vent of FIG. 23, with its actuator partially covering the orifice of the vent;

FIGS. 26A and 26B show a perspective and a side view of another exemplary vent according to the present disclosure, respectively;

FIGS. 26C and 26D show a plan view and perspective view of an exemplary vent cap according to one embodiment of the present disclosure, respectively;

FIG. 27 shows a plan view of another exemplary vent cap according to the present disclosure;

FIG. 28 illustrates how an orifice of a vent may be gradually occluded;

FIG. 29 shows another exemplary vent with an exemplary support structure (actuator not shown);

FIG. 30 shows another exemplary vent with an exemplary support structure (actuator not shown);

FIG. 31 shows a cross-sectional view of the vent of FIG. 30;

FIG. 32 shows a side view of the vent of FIG. 30;

FIGS. 33A, 33B and 33C show another exemplary vent;

FIGS. 34A, 34B and 34C show cross-sectional diagrams of the vent of FIGS. 33A, 33B and 33C, with its actuator in a first, second, and third position, respectively;

FIG. 35 illustrates how a pressure of a breathable gas delivered to a patient varies over time, when an actuator of the vent of FIGS. 33A-C is in different relative positions;

FIGS. 36A, 36b and 36C show cross-sectional diagrams of another exemplary vent with a biasing member, with an actuator of the vent being in a first, second, and third position, respectively;

FIGS. 37, 38 and 39 show diagrams of another exemplary vent, with its actuator in a first, second, third position, respectively;

FIG. 40 shows a cross-sectional diagram of another exemplary vent;

FIG. 41 illustrates an example of a pressure waveform of the breathable gas delivered to the patient when the vent of FIGS. 37-39 is used corresponding to different positions of its actuator;

FIG. 42 shows a plan view of a guiding member and how it may be supported in a vent;

FIGS. 43A, 43B, 43C and 43D show cross-sectional diagrams of another exemplary vent, with its actuator in a range of different positions;

FIGS. 44A, 44B, 44C and 44D show corresponding side views of the vent of FIGS. 43A, 43B, 43C and 43D, respectively;

FIGS. 45A, 45B, 45C, 46A, 46B and 46C illustrate further details of a membrane used with the vent in FIGS. 43A-D and 44A-D and its principles of operation;

FIG. 47A shows another exemplary vent;

FIG. 47B shows a close-up view of a member of the vent of FIG. 47A;

FIG. 47C shows a partial cross-sectional diagram of the vent of FIG. 47A;

FIGS. 48A and 48B show further details of how a member of the vent of FIG. 47A may deform as an actuator moves in different directions;

FIG. 49 shows another exemplary vent, including sealing portions;

FIG. 50 shows a further exemplary vent, including sealing portions;

FIG. 51 shows an exploded perspective view of an exemplary PEEP valve, according to one embodiment;

FIG. 52 shows a side cross-sectional view of the PEEP valve of FIG. 51;

FIG. 53 shows a schematic diagram of an exemplary controller and a pressure differential experienced by the controller;

FIG. 54 shows perspective view of an exemplary pressure regulating device according to one embodiment;

FIG. 55 shows a side cross-sectional view of the pressure regulating device of FIG. 54;

FIGS. 56A-56D illustrate operation of a pressure regulating device when transitioning between PIP and PEEP.

FIGS. 57 and 58 illustrate an example of using an exemplary pressure regulating device with an exemplary CPAP respiratory system;

FIGS. 59A, 59B and 59C show examples of releasable attachment mechanisms which may be used to transition between therapies using a single type of patient interface.

FIGS. 60 and 61 illustrate another example using an exemplary pressure regulating device with the CPAP respiratory system of FIGS. 57 and 58, in combination with a T-piece device.

### DETAILED DESCRIPTION OF EMBODIMENTS

The present disclosure relates to various devices, systems, and methods applicable to a respiratory system arranged to deliver a breathable gas to a patient. In at least one aspect, the present disclosure relates to a vent for use with a respiratory system.

The respiratory therapy mentioned throughout this disclosure can be resuscitation therapy, such as infant or neonate resuscitation therapy, positive airway pressure therapy (PAP), bi-level positive airway pressure therapy, non-invasive ventilation, or another form of respiratory therapy. In some configurations, the system may provide bi-level positive airway pressure therapy to achieve infant resuscitation.

‘Pressure therapy’ as used in this disclosure may refer to delivery of a breathable gas to a patient at a pressure of at least greater than or equal to about 1 cm H₂O. Pressure therapy may be delivered to mimic natural breathing cycles of a patient, and/or delivered in accordance with the patient's breathing cycles to assist with the patient's breathing.

In some configurations, the breathable gas delivered to the patient is, or comprises, oxygen. In some configurations, the breathable gas comprises a blend of oxygen or oxygen enriched gas, and ambient air. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%. In at least one configuration, the gases delivered may be of atmospheric composition. In at least one configuration, the gases delivered may be ambient air.

In relation to infant resuscitation, when in utero, the lungs of a foetus are filled with fluid, and oxygen comes from the blood vessels of the placenta. At birth, the transition to continuous postnatal respiration occurs, assisted by compression of the lungs by the birth canal. Also assisting the infant to breathe is the presence of surfactant that lines the alveoli to lower surface tension. The need for infant resuscitation can occur in a range of circumstances as will be described further below.

Any newborn may require respiratory assistance to either begin or improve breathing at birth. However, several factors may predict the need for resuscitation or respiratory assistance during the transition to continuous postnatal respiration. For example, birth at less than 35 weeks', evidence of significant foetal compromise, maternal infection, or congenital abnormality and emergency caesarean deliveries are associated with an increased need for respiratory assistance at birth.

### Overview

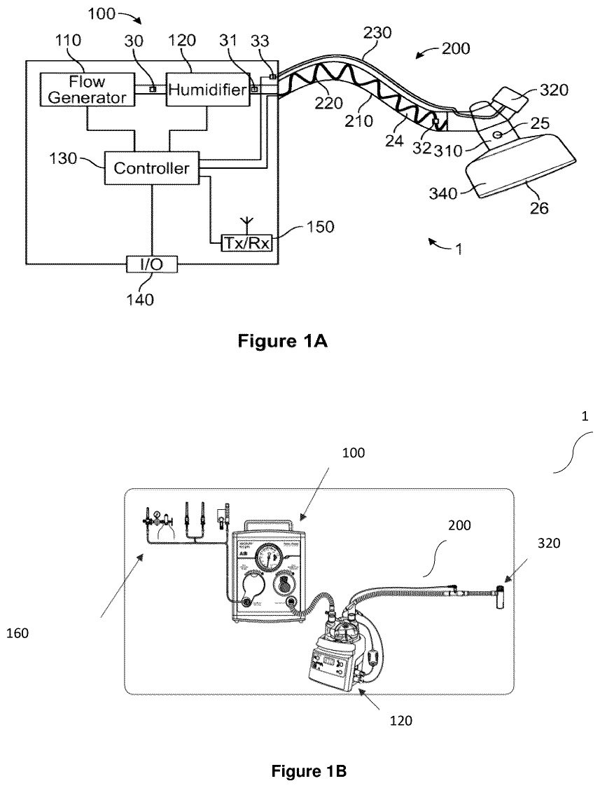

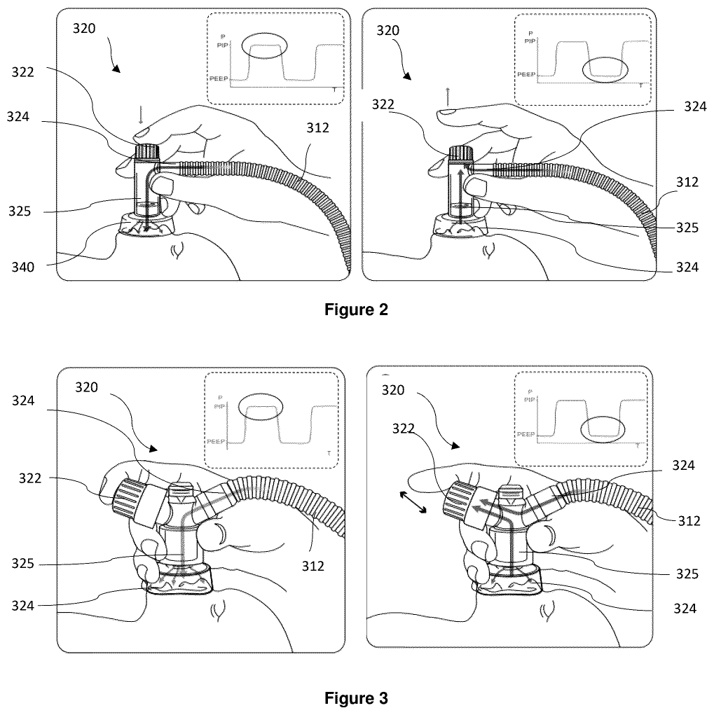

An example of a respiratory system 1 is shown in FIG. 1. Another example of a respiratory system 1 is shown in FIG. 2. The respiratory system 1 is configured to provide respiratory therapy to a patient, by delivering a breathable gas to an airway of the patient.

In general terms, the respiratory system 1 comprises a respiratory apparatus 100, a conduit assembly 200 arranged to convey a breathable gas from the respiratory apparatus 100 to a patient, and a patient interface 340 arranged to be in communication with an airway of a patient. Some embodiments may additionally include a device 320 configured to fluidly connect to the patient interface 340 when delivering respiratory therapy. In at least some embodiments, the device 320 includes suitable connectors allowing it to fluidly couple to an inlet of the patient interface 340 at one end, and fluidly couple to a connector of the conduit assembly 200 at another end.

With reference to FIG. 1, the respiratory therapy apparatus 100 may include a flow generator 110, an optional humidifier 120 for humidifying the gases generated by the flow generator 110, and an associated controller 130 which is configured to control operations of the flow generator 110 and/or the humidifier 120, when present. In one embodiment, the flow generator 110 can be in the form of a blower 110.

The conduit assembly 200 of respiratory system 1 may include a breathing conduit 210 with gases path 24 for guiding the gas from the respiratory therapy apparatus 100 to the patient interface 340. The conduit assembly 200 can include a heating element 220 to heat gas flow passing through the breathing conduit 210 to the patient. The heating element 220 may be in the form of a heater wire or length of conductive wire. The conductive wire may have a predetermined resistance. The heating element 220 can be under the control of a controller, e.g., the central controller 130 or an auxiliary controller.

The respiratory system 1 may include one or more sensors for sensing one or more parameters of the respiratory system 1, such as flow, temperature, humidity and/or pressure. Such sensors can be placed in various locations in the respiratory system 1. One or more sensor outputs can be monitored by the controller 130, to assist in operation of the respiratory system 1.

With further reference to FIG. 1, the respiratory system 1 also includes a user interface 140, comprising, for example, a display and input device(s) such as button(s), a touch screen, or the like. The controller 130 may be configured or programmed to control and/or interact with components of the respiratory system 1, including: operating the flow generator 110 to create a flow of gas (gas flow) for delivery to a patient, receiving one or more inputs from sensors and/or the user interface 140 for reconfiguration and/or user-defined operation(s) of the respiratory system 1, and providing output information (for example on a display) to the user.

The respiratory system 1 may include a transmitter 150, receiver 150, and/or transceiver 150 to enable the controller 130 to receive transmitted signals from the sensors and/or to control the various components of the respiratory system 1. The controller 130 may receive transmitted signals from the sensors related to, or control components including but not limited to the flow generator 110, humidifier 120, humidifier heating element 220, or accessories or peripherals associated with the respiratory therapy apparatus 100 such as the breathing conduit assembly 200. For example, the transmitted signals can relate to, or are processed to instruct control of components. Additionally, or alternatively, the transmitter 150, receiver 150 and/or transceiver 150 may deliver data to a remote server or enable remote control of the respiratory system 1.

The blocks in FIG. 1 represent functional components of the respiratory therapy apparatus 100. It will be appreciated that functionality may be provided by distinct or integrated physical components. For example, the flow generator 110 and the humidifier 120 may be present as an integrated device. An example of a device with integrated flow generator 110 and the humidifier 120 is the Fisher and Paykel Healthcare Airvo™ device. It will also be appreciated that FIG. 1 does not illustrate all functional or physical components or their alternatives of the respiratory system 1. For example, no power supply is depicted in FIG. 1. A person skilled in the art would understand that the respiratory therapy apparatus 100 may include an integrated power supply and/or be connected to an external power supply.

FIG. 2 shows another example of a respiratory system 1, including a respiratory therapy apparatus 100, which may be a positive pressure ventilation device. The respiratory therapy apparatus may be a resuscitator, such as a T-piece resuscitator device. An example of a T-piece resuscitator device is the Fisher and Paykel Healthcare Neopuff™ Infant T-piece Resuscitator. The respiratory therapy apparatus 100 receives a flow of breathable gas from a gas supply source 160 via a gas inlet. The respiratory therapy apparatus 100 maybe connected to an optional humidifier 120 via a gas outlet of the apparatus. The humidified breathable gas is then supplied to the patient from an outlet of the humidifier via a conduit assembly 200 and a device 320, which is connectable to a patient interface (not shown). The gas supply source 160 usually supplies the flow of breathable gas at a constant flow rate to the apparatus 100. The apparatus 100 receives the flow of breathable gas and may be set to vary the pressure of breathable gas delivered to the patient. The apparatus is usually configured in an initial calibration phase to select the level of pressures to be delivered to the patient.

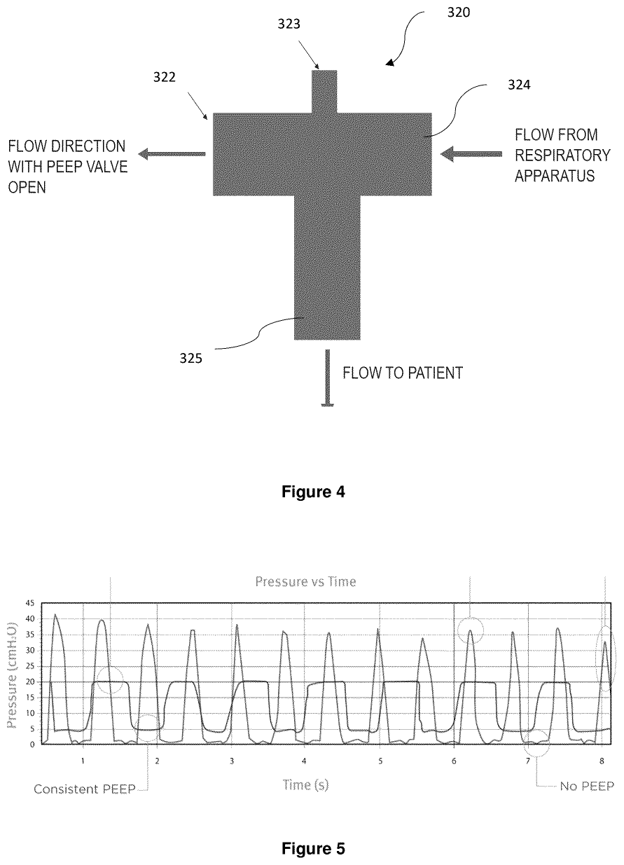

As mentioned above, the device 320 is provided for use with the respiratory system 1, and when in use, it fluidly connects the conduit assembly 200 to the patient interface 340. In some existing respiratory systems, the device 320 is used by an operator of the system to adjust the pressure of gas delivered to the patient, as illustrated in FIGS. 3A, 3B, 4A and 4B.

With reference to FIGS. 3A, 3B, 4A and 4B, each device 320 includes an inlet 324 arranged to receive the breathable gas from the respiratory apparatus 100. An outlet 325 of the device 320 is arranged to be connected to the patient interface 340 when delivering respiratory therapy. Each device 320 also includes a PEEP port 322 arranged to be occluded (i.e., blocked, obstructed, covered, plugged, or otherwise closed) or unoccluded (i.e., unblocked, unobstructed, uncovered, unplugged, or otherwise opened) with an item, for example, a finger or a digit of an operator when delivering respiratory therapy to the patient. When the PEEP port 322 is occluded by the operator as, for example, illustrated in FIG. 3A or 4A, the breathable gas received from the respiratory apparatus 100 is delivered to the patient via the patient interface 340, and the respiratory system 1 delivers the breathable gas at a first pressure to a patient. When the occlusion is removed from the PEEP port 322 as, for example, illustrated in FIG. 3B or 4B, the PEEP port 322 allows gas from within the respiratory system 1 to exit from an internal cavity of the device 320 to ambient air, and the respiratory system 1 delivers the breathable gas at a second pressure to the patient. In this way, resuscitation of a patient can be attempted by varying between the first and second pressures at a selected breathing rate.

FIG. 5 shows flow directions of the breathable gas as it enters the device 320 via its inlet 324, and exits the device 320 from the PEEP port 322 (if it is not occluded), and/or from the outlet 325, which is connected to the patient interface 340 when in use. An optional port, for example, in the form of a duckbill valve 323 may also be included, which can be used for insertion of an auxiliary equipment such as a catheter for fluid clearance or surfactant delivery, and/or a monitoring device for monitoring one or more parameters of inhaled and/or exhaled gas, such as a breath indicator device or a gas detection device, for example, a CO₂ detector for detecting CO₂ in the exhaled gas. Examples of a breath indicator are described in international patent application number PCT/NZ2011/000174 (published as WO 2012/030232) and PCT/IB2019/059813 (published as WO 2020/109915).

The patient interface 340 can be a sealing interface, that is, an interface intended to create a seal with a patient airway. The patient interface 340 can be a mask, including oronasal or nasal mask, cannula such as a nasal cannula, endotracheal tube, or laryngeal mask. The patient interface 340 may be in the form of a CPAP (Continuous Positive Airway Pressure) interface, which may be one or more of mask (nasal or oronasal) or nasal cannula. The patient interface 340 can be held in place on the patient's face by, for example, headgear, and/or by an operator, such as a healthcare professional. It will be appreciated that the patient interface includes a range of different types, sizes and/or fit.

A neonatal interface may be any interface, such as described above, that is configured for use with an infant or neonate. The neonatal interface may be configured to at least partially, and preferably substantially seal around the nose and/or mouth of the patient.

To provide respiratory therapy to a patient, pressure delivered to the patient may be regulated to mitigate or prevent injury to patients. This is particularly relevant to infants and neonates due to the fragility of their lungs and airway. The PEEP port 322 may include a pressure regulating valve which actuates at a level of pressure, namely, at a set PEEP, to allow the gas from within the respiratory system 1 to vent externally and reduce the excess pressure within the respiratory system 1. In other words, the PEEP port 322 may include a PEEP valve. Another pressure regulating valve may be provided in the respiratory apparatus 100, to control the pressure of the breathable gas delivered to the patient at PIP. In addition, a maximum pressure relief valve may also be provided in the respiratory apparatus 100, to set the maximum pressure relief that may be delivered to the patient.

In some embodiments, the first pressure level may be delivered at or near the patient terminal end 26 (as shown in FIG. 1) at a first time or during a first time window. The first pressure level may be delivered at or near the patient terminal end 26 once interface fit is confirmed.

Similarly, a second pressure level can be delivered at or near the patient terminal end 26 at a second time or during a second time window. The second pressure level may be delivered at or near the patient terminal end 26 once interface fit is confirmed and/or once intended second pressure level has been confirmed in the resuscitator, for example, by sealing the outlet of the device with a protective cap. The respiratory system 1 continuously provides the breathable gas to the patient at the first and the second pressure levels in order to mimic patient's breathing cycles. Typically, 30-60 breathing cycles per minute are provided to the patient during respiratory therapy. In some applications, a patient's breathing cycles are manually determined by a clinician. It should be appreciated that the number of breathing cycles per minute required is largely dependent on the type of therapy to be provided to the patient, patient's condition (age, breathing condition, etc.), and usually varies from patient to patient.

In one embodiment the first pressure level is equal to target PIP or desired PIP. In some examples, the first pressure may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O, and a useful value may be selected between any of these ranges (for example about 15 to about 60, about 20 to about 25, about 21 to about 30, about 21 to about 27, about 21 to about 25, about 22 to about 30, about 22 to about 29, about 22 to about 25, about 23 to about 30, about 23 to about 28, about 23 to about 26, about 24 to about 30, about 24 to about 29, about 24 to about 28, about 24 to about 26 or about 25 to about 30 cm H₂O).

A higher PIP may be needed for first few breathing cycles (for clearing liquid from airways and beginning lung aeration) and/or if the patient does not respond positively to initially given respiratory therapy. In addition, the level of pressure required for resuscitation may vary from patient to patient, depending on factors such as maturity of lungs, presence of lung disease, disorder, and similar. The pressure values and/or ranges mentioned above are for guide only and in practice target pressures can be individually adjusted depending on for example, patient's response, patient requirements and/or clinician preference.

In one embodiment, the second pressure level is equal to target PEEP or desired PEEP. In some example, the second pressure may be 1, 2, 3, 4, 5, 6, 7, 8, 9, 10, 11, 12, 13, 14 or 15 cm H₂O, and a useful value may be selected between any of these ranges (for example, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 2 to about 8, about 2 to about 6, about 2 to about 5, about 3 to about 8, about 3 to about 5, about 4 to about 8, about 4 to about 7, about 4 to about 5, about 5 to about 8 or about 6 to about 8 cm H₂O). The second pressure may be about 5 cm H₂O, but can be set depending on factors as outlined above. The pressure values and/or ranges mentioned above are for guide only and in practice target pressures can be individually adjusted depending on, for example, patient's response, patient requirements and/or clinician preference.

The configuration of the devices 320 described may allow for one handed operation during respiratory therapy.

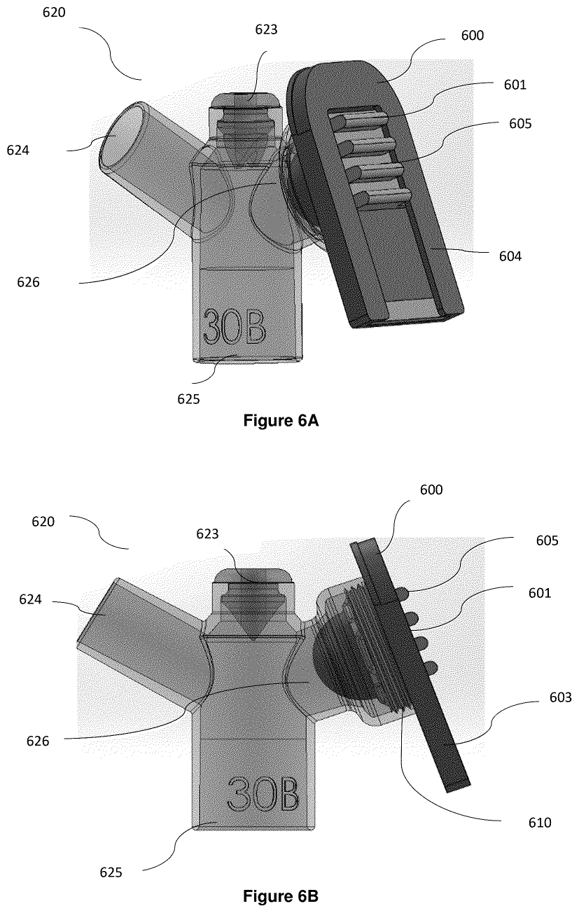

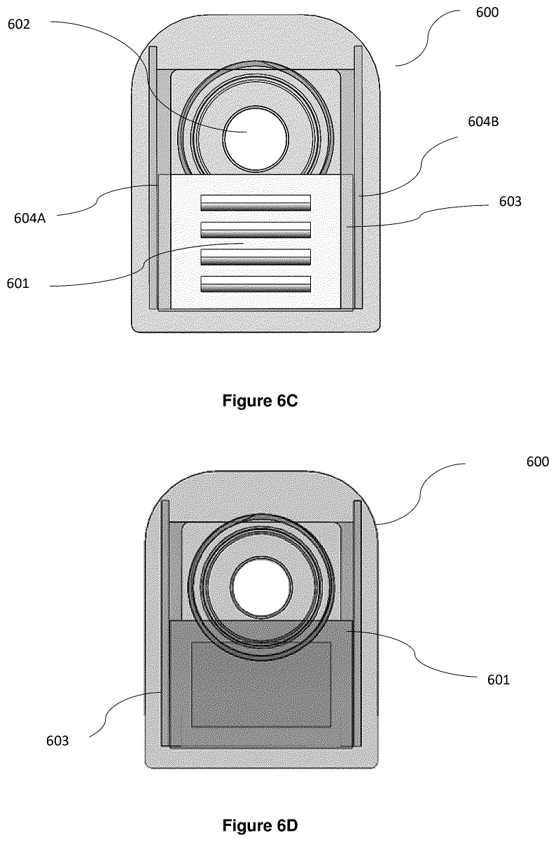

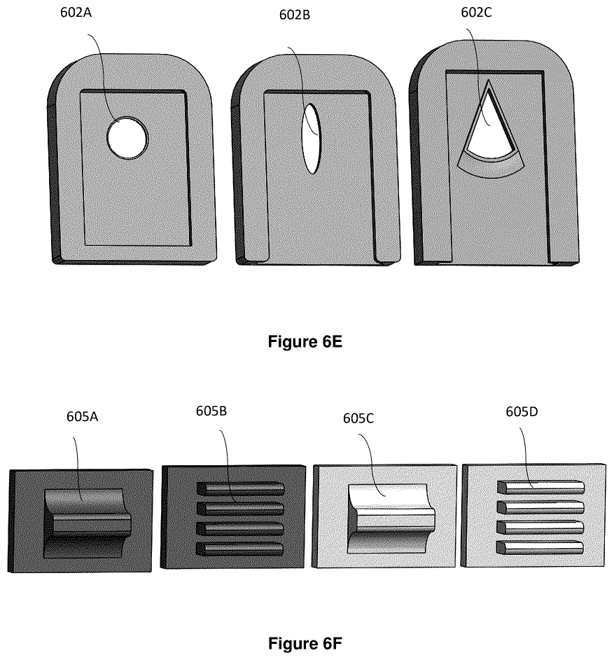

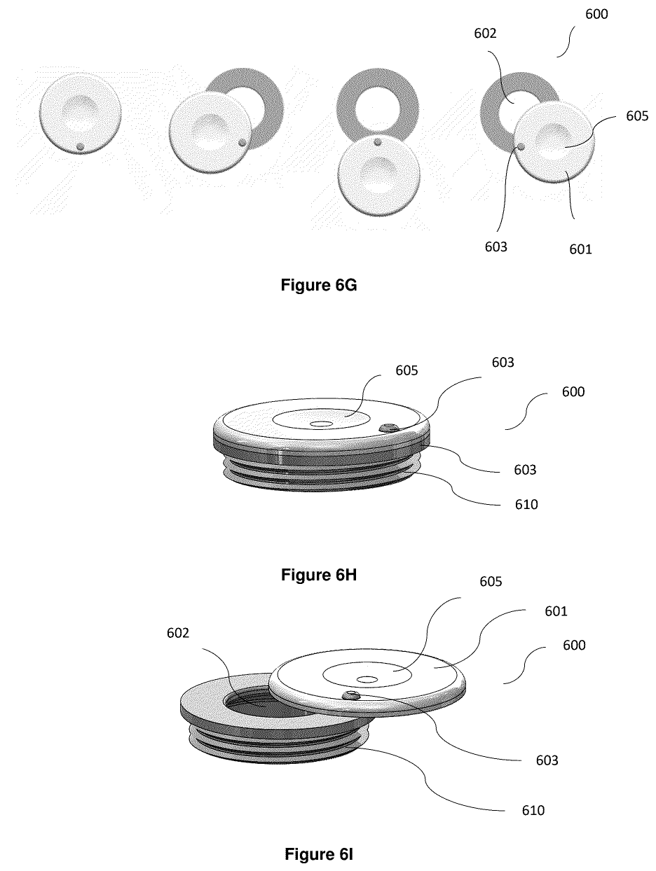

Overtime, the varying pressures of breathable gas conveyed to the patient may be represented by a roughly square waveform, as, for example, shown in the insets of FIGS. 3A, 3B, 4A and 4B, respectively, in which the vertical axis represents pressure (P) while the horizontal axis represents time (T). FIG. 6 also illustrates an exemplary waveform of pressures over time 5001 provided by the devices 320 as configured according to FIGS. 2-4. The square shaped waveform 5001 indicates that there is usually a rapid change in pressure, i.e., indicated by the sharp transition slope between target PEEP and target PIP. It may be desired, at least in certain circumstances or applications, to provide a relatively or comparably smooth transition between different target pressures.

According to the present disclosure, a vent may be provided for use with the respiratory system 1 described above. The vent includes a movable actuator, wherein movement of the actuator adjusts an area of the vent available for gas from within the respiratory system 1 to exit the respiratory system 1 via the vent. Accordingly, the vent allows a greater level of control over a pressure waveform of the breathable gas delivered to the patient, especially when the respiratory system 1 switches between different pressure settings, and may assist the respiratory system 1 to provide a relatively smooth transition between different target pressures.

In some embodiments, the vent is configured to be retrofitted to the PEEP port 322 of the device 320 through a suitable connection mechanism. For example, complimentary threaded portions may be provided in both the vent and near the PEEP port 322. In this example, the vent couples to the device 320 via these threaded portions in use, and allows a gradual and/or stepped closing or opening of a gas flow path via the PEEP port 322 and the vent. In some embodiments, the device 320 may be configured such that it does not include a PEEP port 322 similar to what is shown in FIGS. 2 and 3. Instead, the vent is directly coupled to an air outlet of the device and replaces the PEEP port 322.

The respiratory therapy may be a pressure therapy delivered to a patient to assist with breathing and/or treat breathing disorders. The pressure therapy may involve the respiratory system 1 providing a breathable gas to the patient at one or more target pressures for one or more time windows. The vent allows the respiratory system 1 to provide a relatively smooth transition when switching between different target pressures. That is, a range of intermediary pressures may also be provided to the patient for one or more time windows.

In some embodiments of the present disclosure, a pressure waveform of the breathable gas conveyed to the patient may be substantially symmetrical, meaning a pressure increase and a pressure decrease follows a substantially symmetrical waveform. Providing the substantially symmetrical pressure waveform requires a gradual increase of pressure and a gradual decrease of the pressure to occur in similar time durations. In some embodiments of the present disclosure, a pressure waveform of the breathable gas conveyed to the patient may be asymmetrical, meaning a gradual increase of the pressure and a gradual decrease of the pressure may occur in different time durations. In other embodiments, the vent may be configured such that the pressure is increased more rapidly, than it is decreased. In some embodiments, the vent may be configured such that the pressure is decreased more rapidly, than it is increased.

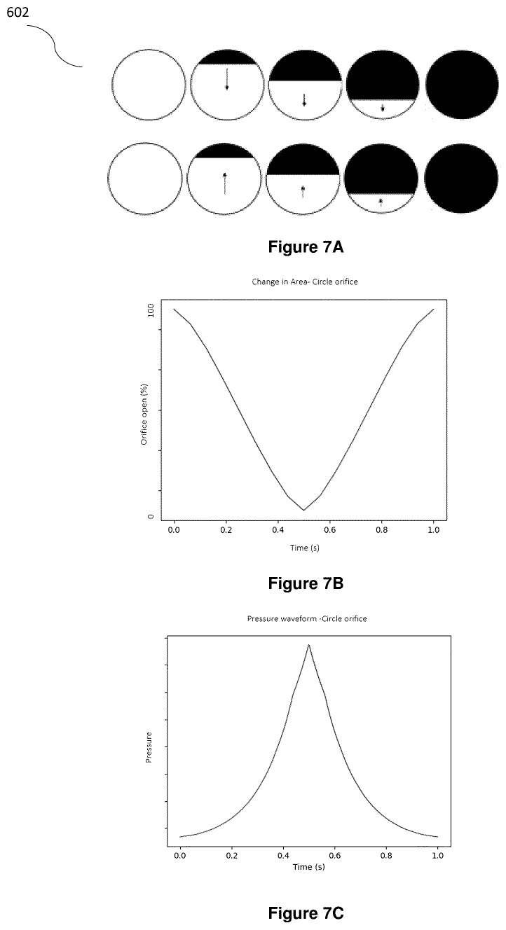

Various embodiments of the vent will now be described with reference to FIGS. 7A to 50.

In general terms, the vent allows gas from within the respiratory system to exit to ambient air via the vent. The vent comprises a movable actuator, for example, operated by an operator, and movement of the actuator adjusts an area of the vent available for the gas to flow through. In doing so, the pressure of the breathable gas conveyed to the patient is gradually adjusted. The rate of change of pressure depends at least in part on the speed at which the operator is delivering breaths to the patient, as well as how fast the operator is moving the actuator.

Adjusting the area of the vent available for the gas to flow through controls the resistance to flow of gases exiting the vent, which in turn controls the pressure of the breathable gas delivered to the patient. For example, when the actuator is moved to reduce the area of the vent, the resistance to flow through the vent is increased, which in turn will increase the pressure delivered to the patient for a given, constant flow rate of gases through the respiratory system 1 within a time period. When the actuator is moved to increase the area of the vent, the resistance to flow through the vent is decreased. This in turn decreases the pressure delivered to the patient, for a given constant flow rate of gases through the respiratory system 1 within a time period.

When movement of the actuator results in full occlusion or closure of the vent and there is no area of the vent available for gas to flow through, a flow of gases may exit elsewhere in the system 1. The flow of gases may exit through another vent or outlet included in respiratory apparatus 100. For example, the flow of gases may exit via a PIP vent or PIP valve, which actuates when pressure in the system 1 increases to PIP due to full occlusion/closure of the vent by the actuator.

In some embodiments, the actuator is movable to cause a gradual and/or stepped occluding or unoccluding of an air outlet of the respiratory system, for example a PEEP port of a T-piece device, such that the pressure of the breathable gas delivered to the patient is gradually changed or changed in steps. In some embodiments, the actuator is movable to cause a gradual and/or stepped opening or closure of one or more orifices or openings of the vent, thereby adjusting the pressure of the breathable gas delivered to the patient.

In some embodiments, the movement speed of the actuator is predominantly determined by the operator. In other embodiments, the movement speed of the actuator may be at least in part determined by a configuration of the vent, as will be described below.

Although the embodiments below are described with reference to accompanying figures which show certain combinations of various features of the vent, it should be appreciated that features from different embodiments may also be combined, and not necessarily restricted to what is shown in the accompanying figures.

FIGS. 7A to 50 show various embodiments of a vent including a movable actuator.

FIGS. 7A and 7B show a first exemplary vent 600 including a movable actuator 601, connected to a device 620 of the respiratory system 1. The device 620 includes an inlet 624, arranged to receive a flow of breathable gas from the respiratory apparatus 100. A first outlet 625 of the device 620 is connectable to a suitable patient interface (not shown) to deliver the breathable gas to the patient. An optional port, for example in the form of a duckbill valve 623 is also included in the device 620 for insertion of an auxiliary equipment such as a catheter for fluid clearance or surfactant delivery to the patient, and/or a monitoring device for monitoring one or more parameters of inhaled and/or exhaled gas, such as a breath indicator device or a gas detection device, for example, for detecting CO₂ in the exhaled gas.

In this example, the vent 600 is connected to a second outlet 626 of the device 620, via one or more suitable connectors. The second outlet 626 may be a PEEP port of the device 620 as described above. In some embodiments, for example as illustrated in FIGS. 7A and 7B, the connectors may include complimentary threaded portions 610 provided in both the vent 600 and the device 620. In other embodiments, the connectors may be in other forms, for example, detachable connectors and/or interchangeable connectors. In other embodiments, the vent 600 may be integrally formed with the device 620. While delivering respiratory therapy, the actuator 601 may be slid, for example, by the operator to adjust an area of the vent 600 available for the gas to pass through and exit from the respiratory system 1, which in turn controls a pressure of the breathable gas delivered to the patient.

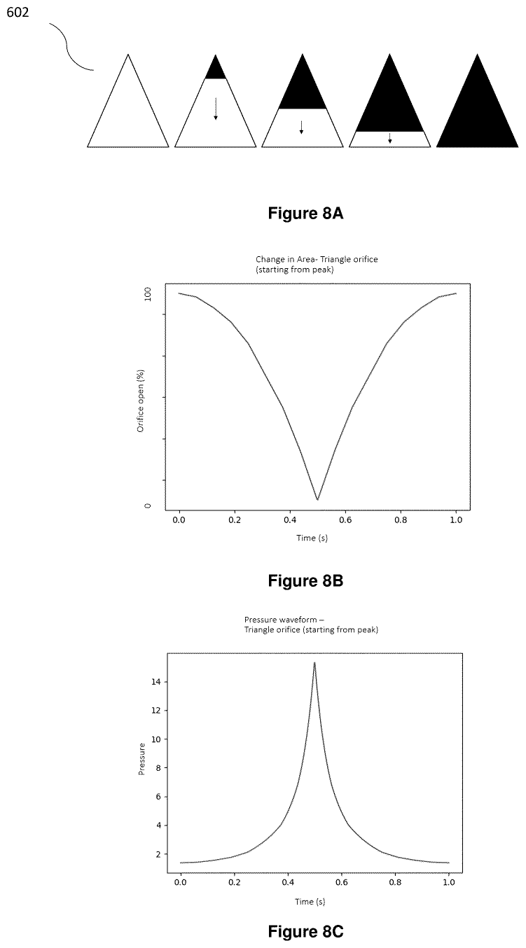

FIGS. 8A and 8B show another exemplary vent 600 including a slidable actuator 601. The device 620 is omitted from these figures for ease of illustration. As indicated, the slidable actuator 601 can be movable, for example, by a finger of the operator, to cover, partially cover or uncover an opening, for example, in the form of an orifice 602 of the vent 600. The area of the vent 600 available for the gas to flow through to exit the respiratory system 1 is determined based on the uncovered area of the orifice 602. The vent 600 may additionally include a support structure 603, and the actuator 601 is movably coupled to and supported by this support structure 603. The support structure 603 includes one or more channels for retaining and guiding the sliding movement of the actuator 601.

FIGS. 7A and 7B show the exemplary vent 600 using a single U-shaped channel 604 which extends a peripheral portion of support structure 603. The peripheral portion may include a left, a top, and a right side of the support structure 603. FIGS. 8A and 8B show another exemplary vent 600 which includes two substantially parallel channels 604A, 604B, disposed on either side of the orifice 602. The two parallel channels 604A, 604B may be disposed on a surface of the support structure 603. The channels 604, 604A, 604B receive a left and a right edge of the actuator 601, to help guide the sliding movement of the actuator 601. These channels may also assist with maintaining a relative position of the actuator 601 and the support structure 603, so that the actuator 601 does not become accidentally disengaged from the support structure 603 in use.

As shown in FIG. 8A, when the slidable actuator 601 is slid to a lower end of the vent 600, the orifice 602 is entirely open, allowing gas from within the respiratory system to flow through the orifice 602, to ambient air. As the actuator 601 is slid towards an upper end of the vent 600, the orifice 602 is gradually covered by the actuator 601. The gradual covering of the orifice 602 reduces the area of the vent 600 available for the gas to flow through, thereby elevating the pressure of the breathable gas conveyed to the patient. Similarly, the orifice 602 is gradually uncovered by the actuator 601, as it is slid towards the lower end of the vent 600. The gradual uncovering of the orifice 602 increases the area of the vent 600 available for the gas to flow through, which reduces the pressure of the breathable gas delivered to the patient.

In some embodiments, when the orifice 602 is fully or substantially covered by the actuator 601, the pressure of the breathable gas delivered to the patient corresponds to PIP, or positive inspiratory pressure. When the orifice 602 is fully or substantially uncovered, the pressure of the breathable gas delivered to the patient corresponds to PEEP, or positive end expiratory pressure. When the orifice 602 is partially covered, pressures between PEEP and PIP are delivered to the patient. As the actuator 601 is repeatedly moved up and down the vent 600, different pressure levels of breathable gas is supplied to the patient, in accordance with the need of the patient.

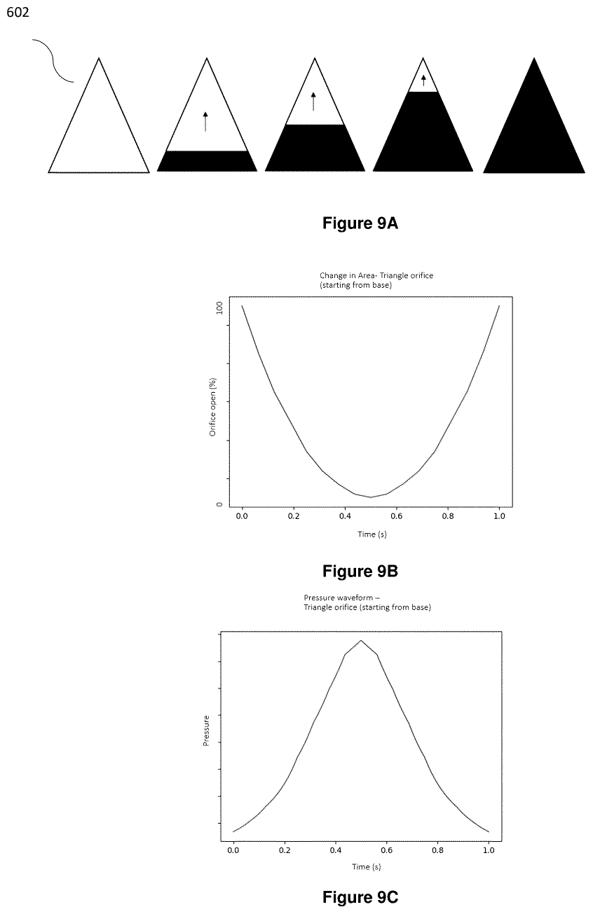

In FIGS. 8A and 8D, the orifice 602 forms a substantially circular shape. It should be appreciated that the orifice 602 can be provided in various other shapes and configurations, for example, as shown in FIG. 9, and not limited to the circular shape shown in FIGS. 8A and 8D. In addition, a single orifice 602 is provided in the vent 600 illustrated in FIGS. 8A, 8B and 9. It should be appreciated that a plurality of orifices 602 may alternatively be provided in the vent 600. In at least some embodiments, the configuration of the orifice 602 may be determined at least in part on a desired rate of change of covering or uncovering of the orifice 602 during movement of the actuator 601. The configuration of the orifice 602 may also be determined at least in part on inspiration to expiration ratio (I:E) to the patient. In some embodiments, the configuration of the orifice 602 may be determined at least in part on a desired shape of the pressure waveform of the breathable gas to be delivered to the patient over time.

FIG. 9 shows examples of a vent 600 including three exemplary orifices, each having a different shape. For ease of illustration, the actuator 601 is omitted from these embodiments. As indicated, the orifice 602 may be provided in a circular shape as indicated by 602A, an oval shape as indicated by 602B, or a cone shape as indicated by 602C. With these orifices, as the actuator 601 is moved at a roughly constant speed by the operator, a rate of change in the open area of the orifice varies along a movement direction, which is likely to result in a non-linear change in the pressure of the breathable gas delivered to the patient.

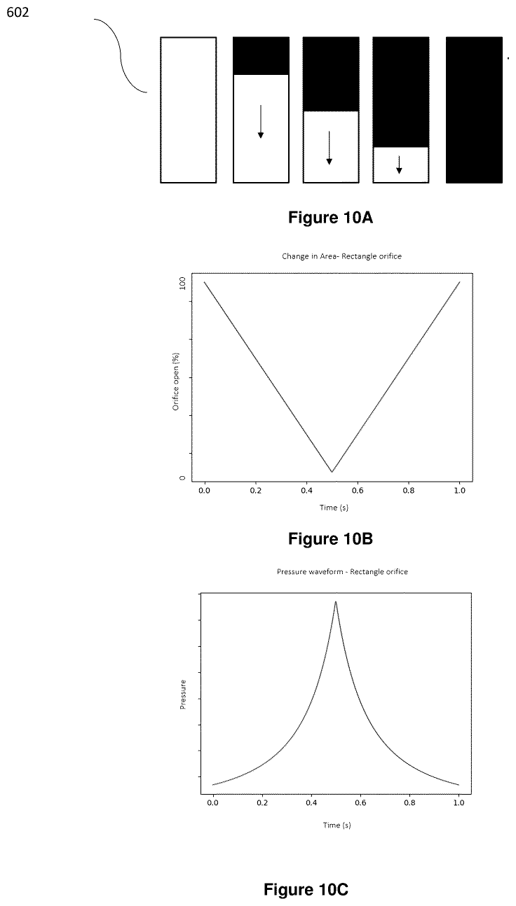

FIG. 10 indicates how a circular orifice 602 may be gradually covered and uncovered in a breathing cycle. The dark coloured areas (e.g., represented by 1001, 1003, 1005 and 1007) within circular shape 602A indicate covered area of the circular orifice 602 by the actuator 601, and the light coloured areas (e.g., represented by 1002, 1004, 1006 and 1008) within the circular shape 602A indicate open area of the orifice 602 which allows gas from within the respiratory system 1 to flow through. With the movement of the actuator 601, for example, along a movement path represented by the arrow in FIG. 10, the circular orifice 602 is fully covered as illustrated by 1007. In will be understood that movement of the actuator 601 along an opposite direction to the movement path shown in FIG. 10 would gradually cover the circular orifice 602.

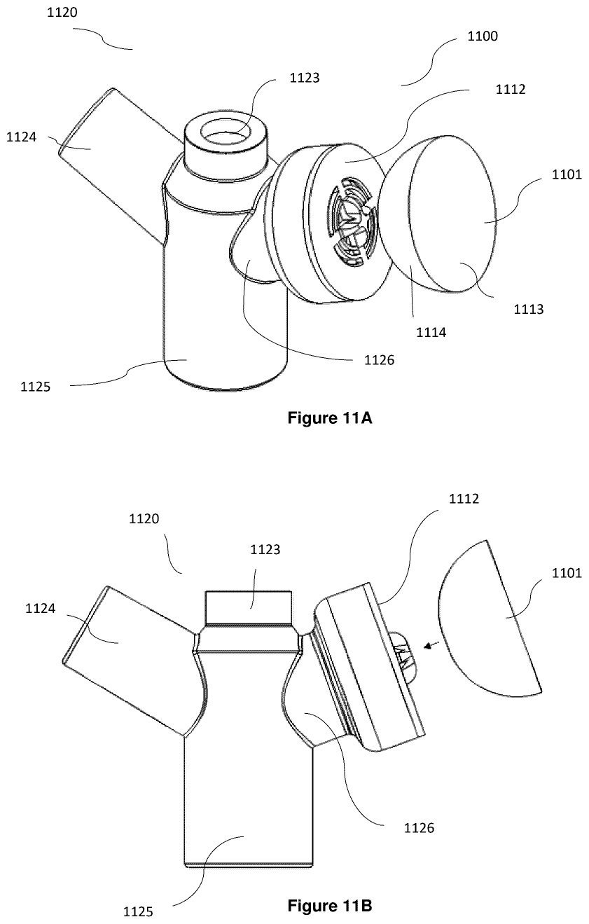

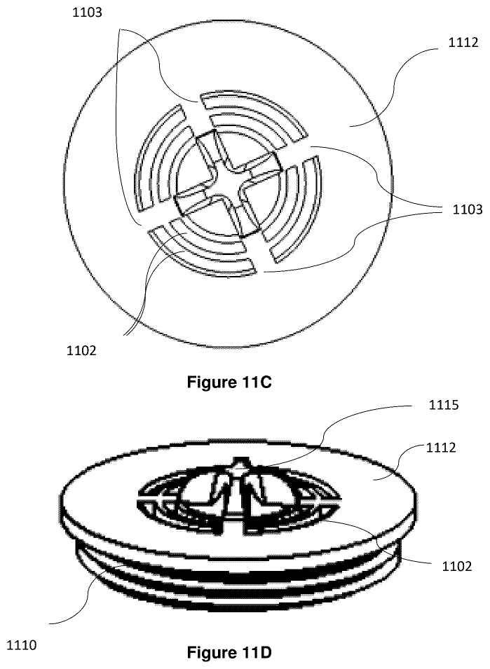

FIG. 11 is an exemplary diagram showing how the open area of the orifice 602 changes as the actuator 601 is moved to different positions relative to the orifice 602. If the actuator 601 is moved at a constant speed, along its movement path, the open area of the orifice 601 changes in a non-linear manner from a substantially fully open state at time T1, to a substantially full closed state at time T2, and again to a substantially fully open state at time T3, indicated by the curve 1111 in FIG. 11. A corresponding waveform 1211 of the pressure delivered to the patient as a result of movement of the actuator 601 is illustrated in FIG. 12. As described above, the pressure delivered to the patient is at the lowest level at time T1 and T3 when the orifice 602 is substantially fully opened. The curve 1211 of FIG. 12 demonstrates a smoother transition between a low and a high pressure provided to the patient. Comparing the pressure waveform 1211 of FIG. 12 and the pressure waveforms shown in FIGS. 3A, 3B, 4A, 4B and 6, it can be observed that the pressure increase or decrease is more gentle and smooth, which may be desirable for some applications.

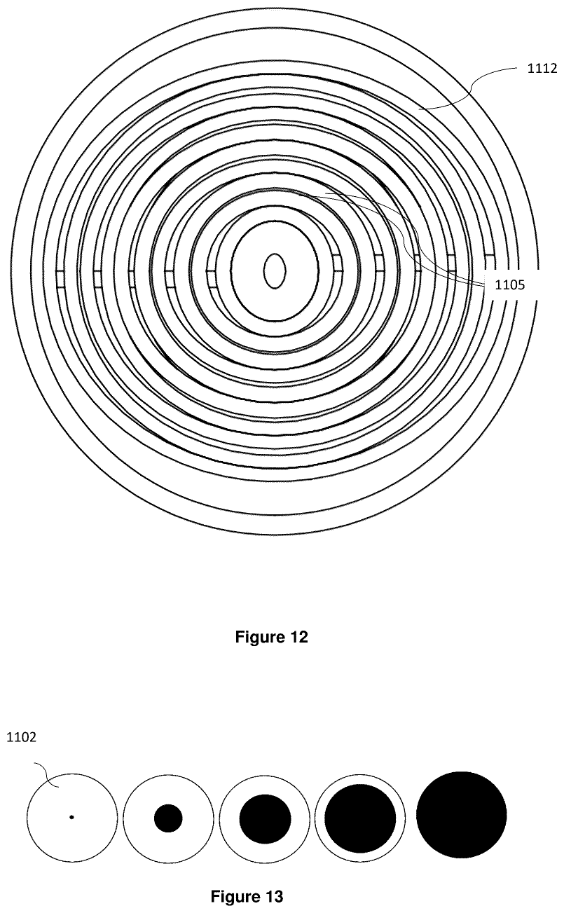

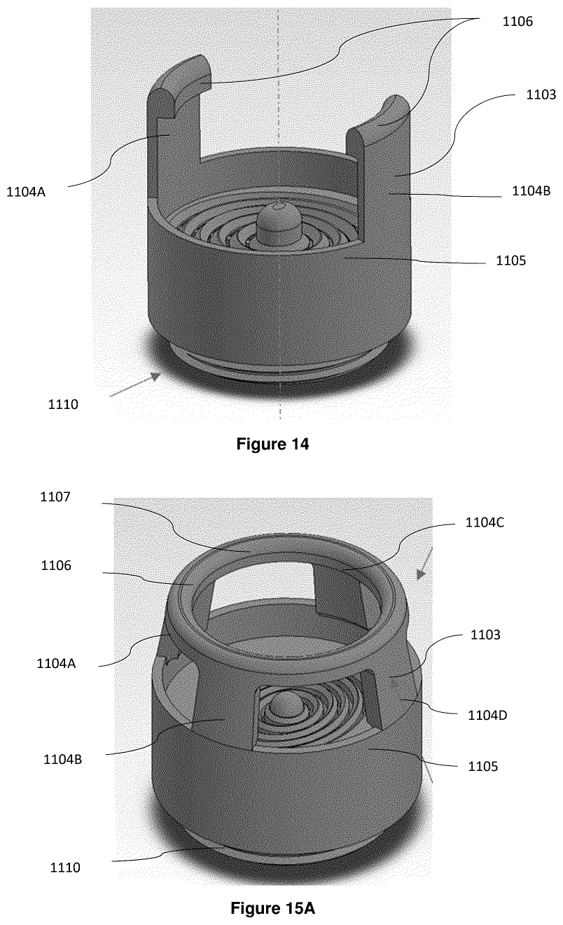

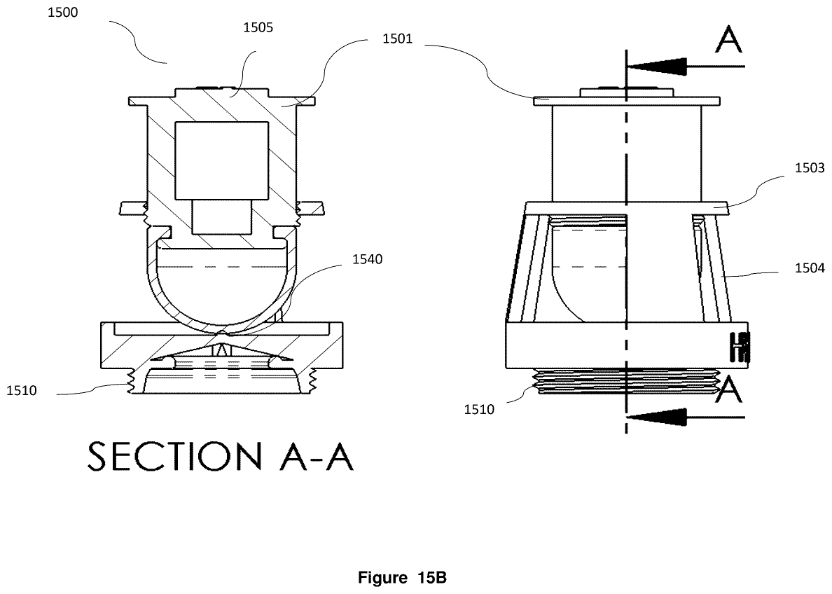

Similarly, FIG. 13 shows how a triangular shaped orifice 602 is gradually covered by the actuator 601 in use. When the actuator 601 is moved to cover an apex 602D of the orifice 602, a reduction of the open area of the orifice 602 is relatively small. However, as the actuator 601 moves to approach a side 602E opposite to the apex 602D (i.e., base 602E) of the triangular shaped orifice 602, the reduction of the open area 602 becomes more significant with each incremental movement of the actuator 601. This is reflected in FIG. 14, which shows how the open area of the triangular shaped orifice 602 changes as the actuator 601 is moved to cover the triangular shaped orifice 602 from the apex 602D to the base 602E and then to open the triangular shaped orifice 602 again from the base 602E to the apex 602D. FIG. 15 shows a pressure waveform of the breathable gas delivered to the patient corresponding to the open area waveform of FIG. 14 with the use of the triangular shaped orifice 602. As the actuator 601 moves to or away from the bottom (i.e., 602E in this example) of the triangular orifice 602, the pressure changes more rapidly, compared to when the actuator moves to or away from the apex (i.e., 602D in this example) of the triangular orifice 602.

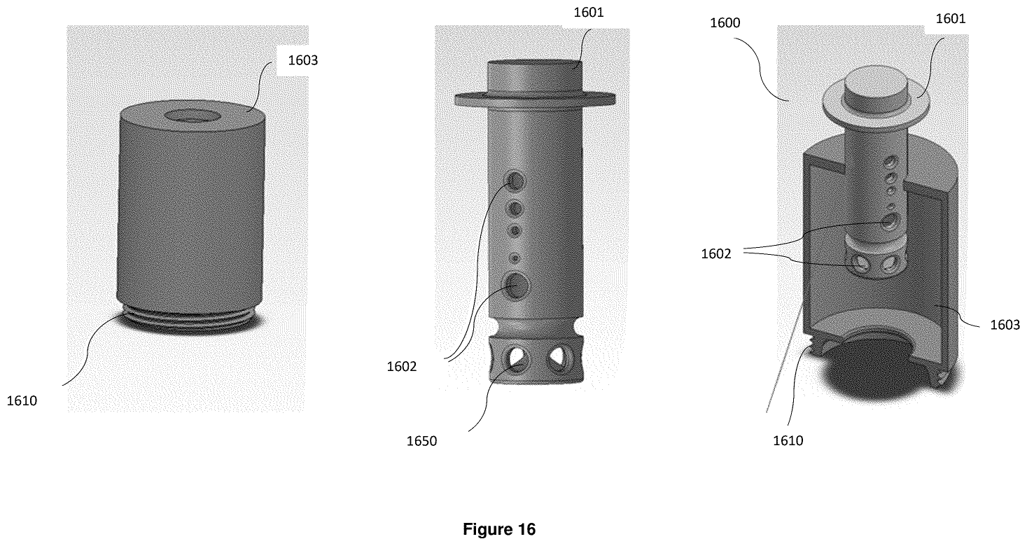

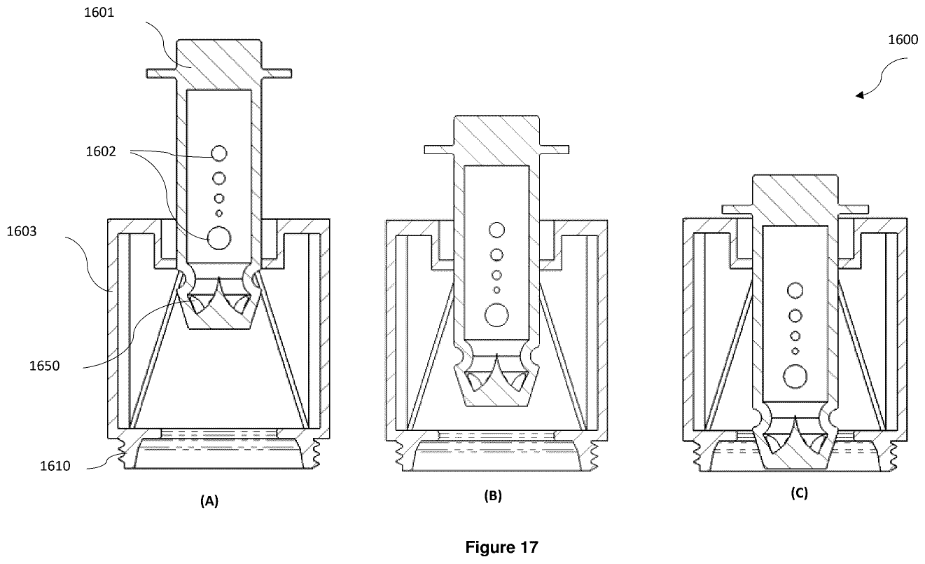

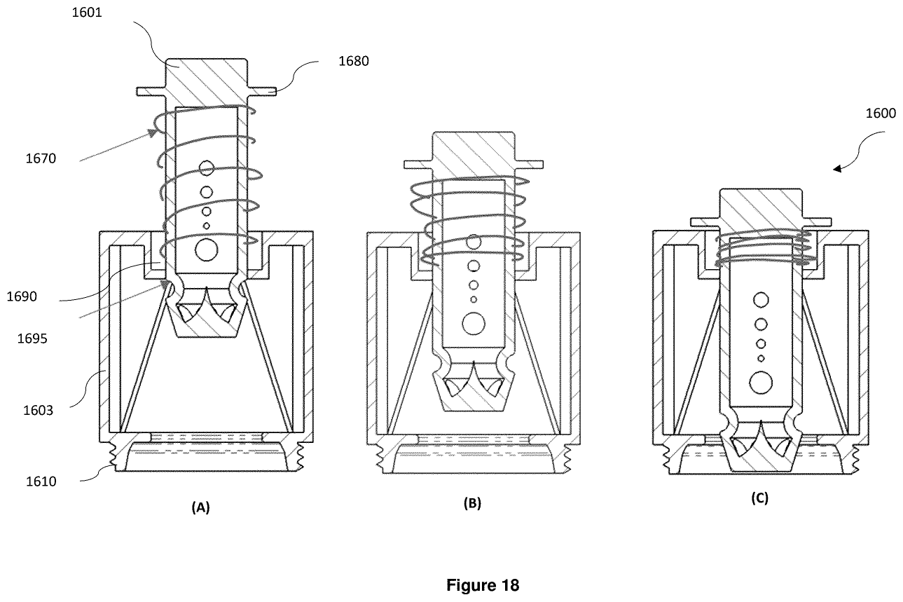

FIGS. 16 to 18 demonstrate how a pressure waveform may be different, even with the same orifice 602 configuration. The same triangular orifice 602 as shown in FIG. 16 is used, however, the actuator 601 is moved to cover the orifice 602 gradually from the base 602E (instead of from the apex 602D as shown in FIG. 13). In this instance, the rate of change of the open area as shown in FIG. 17 (and therefore the corresponding pressure delivered to the patient as shown in FIG. 18) is greater when the actuator 601 is moved away from or moved towards the base (e.g., 602E) of the triangular orifice 602, and is slower as the actuator 601 is moved towards or away from the apex (e.g., 602D) of the triangle orifice 602.

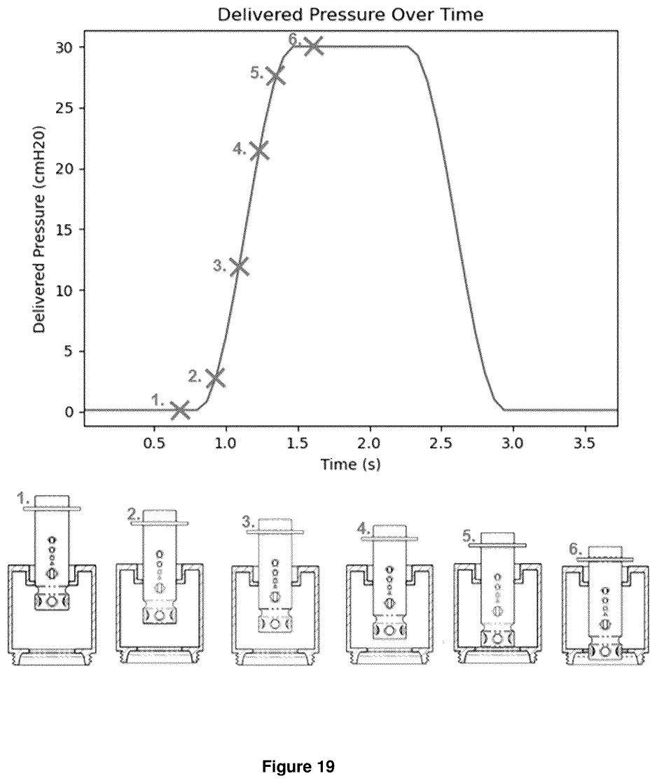

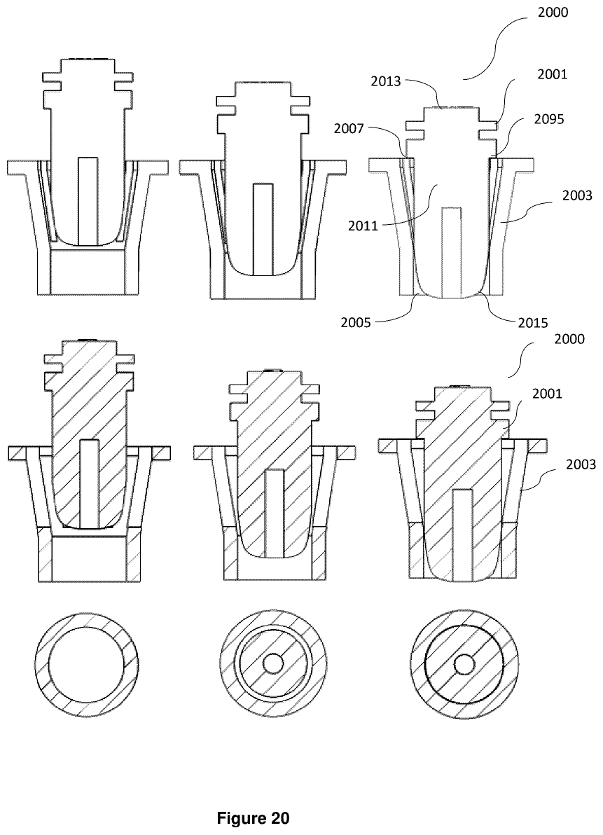

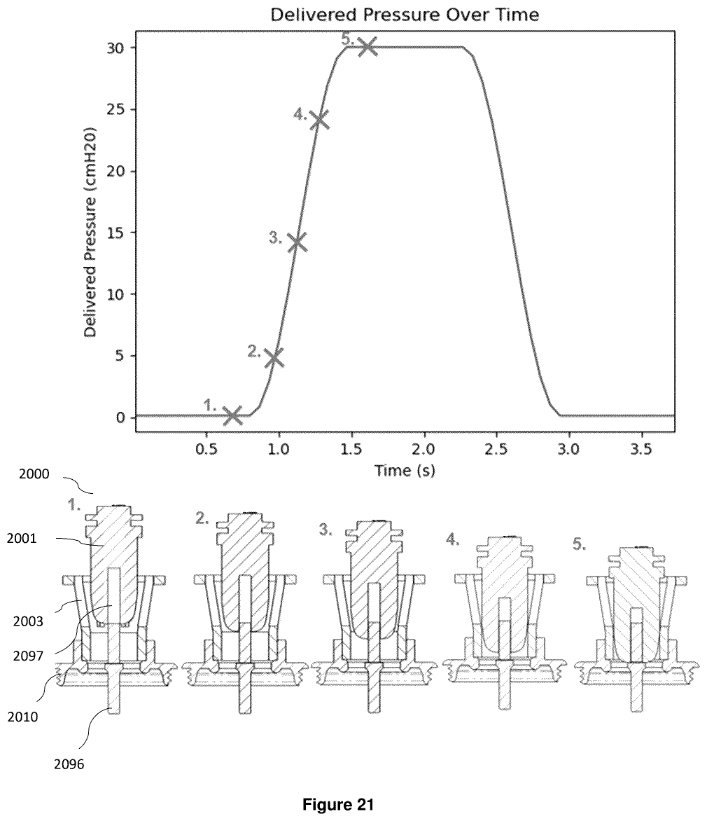

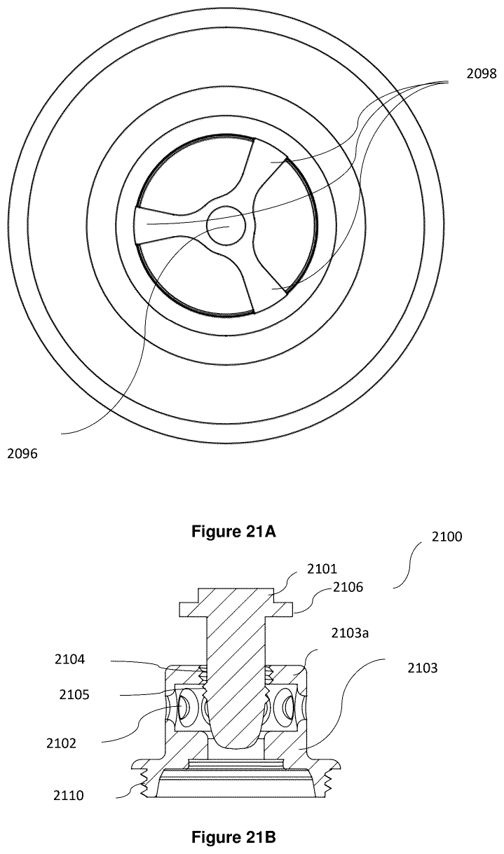

In some embodiments, it is possible to configure the orifice 602 such that as the actuator 601 is moved at a constant speed, a rate of change of the open area of the orifice 602 also roughly remains constant. FIG. 19 shows an example of how this may be achieved, by using a rectangular shaped orifice 602. As the actuator 601 is moved to gradually cover or uncover the orifice 602, the rate of change of the open area remains constant, as represented by the straight line in FIG. 20. FIG. 21 shows a corresponding pressure waveform resulted from using a rectangular shaped orifice. The pressure delivered to the patient still follows a more gradual and smooth curve, rather than a sudden increase or decrease as in previous systems as shown in FIGS. 3A, 3B, 4A, 4B and 6.

Referring back to FIGS. 7A and 7B, in some embodiments, the actuator 601 includes an engaging member 605 arranged to be engaged by, for example, a finger or digit of the operator when operating the actuator 601. The engaging member 605 can include a formation which increases a frictional force between the finger of the operator and the actuator 601 and/or influence distribution of force on the actuator 601. The formation may simply be a rough surface texture. Alternatively or additionally, the formation may include, for example but not limited to, a protrusion, button, one or more ridges, a depression, recess, groove, indent and similar thereof to allow the operator to easily move the actuator 601 when in use. The formation provides tactile feedback so that an operator of the system knows their finger is located correctly on the actuator of the vent. This may be useful if the operator needs to look elsewhere while operating the system (e.g. monitoring conditions or reactions of the patient).

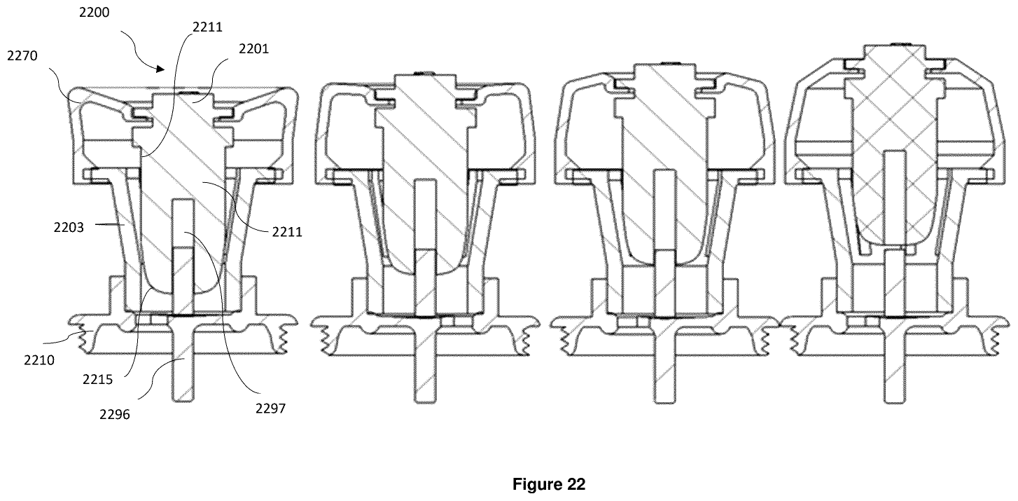

FIG. 22 shows various examples of the actuator 601 including different types of engaging members 605A, 605B, 605C, 605D. It will be appreciated that the actuator 601 and the support structure 603 and its engaging member 605 may be made from the same materials, or different materials, including plastic, foam, rubber, and similar thereof, depending on friction of movement, user comfort, sealing ability, and so on. In addition, the actuator 601 may be configured as a transparent or an opaque component of the vent 600, such as illustrated in FIG. 22. The actuators 605C and 605D are made from a clear plastic and is substantially transparent. Other actuators 605A and 605B are made from dark coloured materials which may be opaque. Similarly, the support structure 602 may also be made from clear or opaque materials.

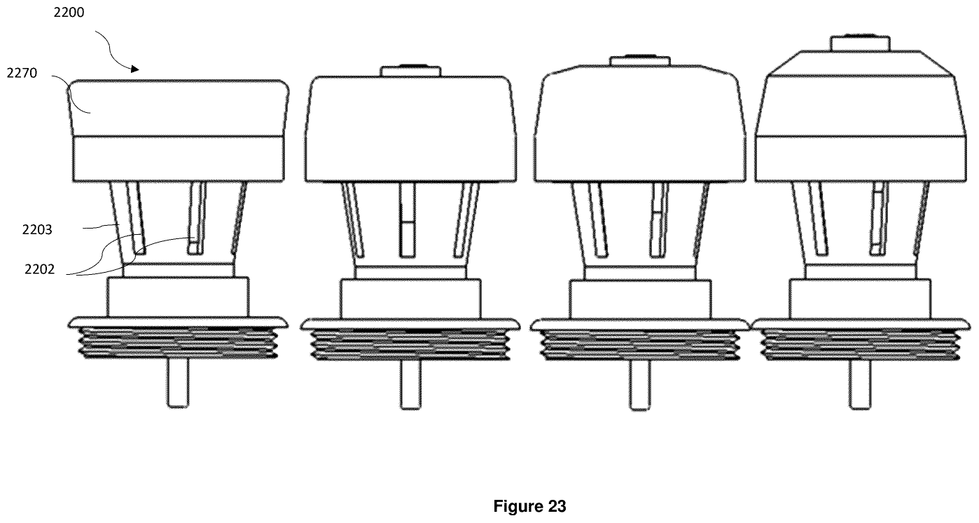

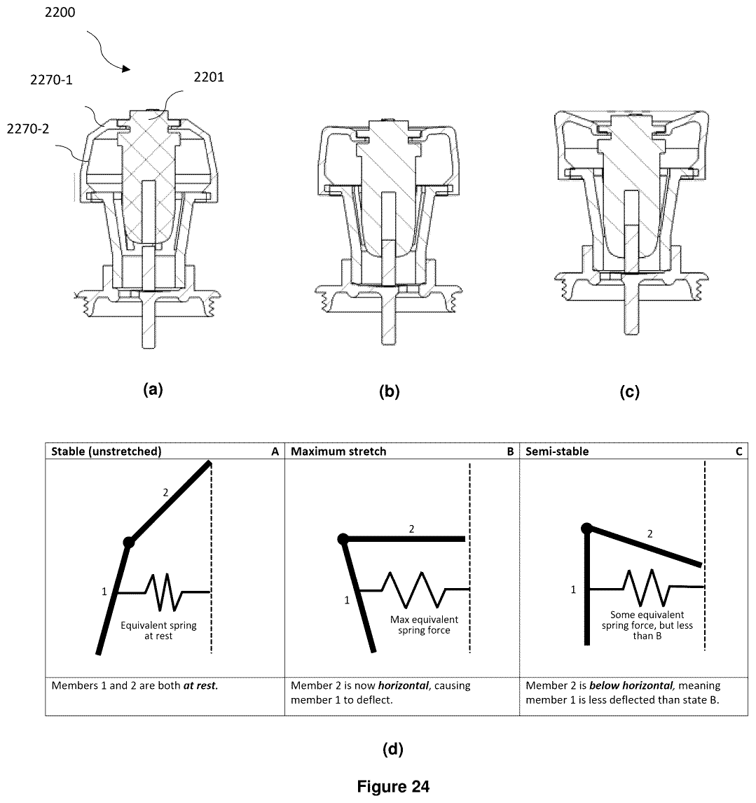

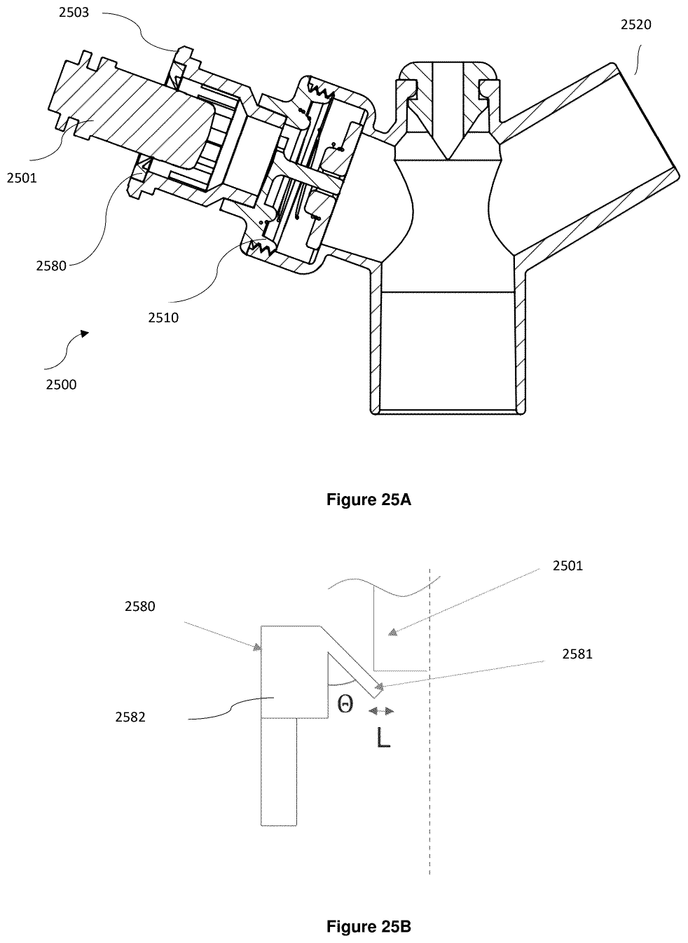

FIG. 23 shows another exemplary vent 600 including a movable actuator 601, with the actuator 601 moved to a range of different positions. As shown, the actuator 601 is again moved to occlude or unocclude an orifice 602 of the vent 600. The support structure 603 of this embodiment includes a hinge 603, and the actuator 601 is rotatable with respect to the hinge 603, for example, as moved by an operator. In this example, an engaging member 605 may be formed as a circular indent located in a centre region of the actuator 601, allowing the operator to place the finger on when operating the vent 600. FIG. 24 shows a side perspective view of the vent 600 of FIG. 23 in a first state, when the actuator 601 is moved to a position where it fully occludes the orifice 602. FIG. 25 shows another side perspective view of the vent 600 of FIG. 23 in a second state, when the actuator 601 is rotated to a different position where the orifice 602 is partially occluded. Connector portions, such as threaded connectors 610 may be formed on a lower surface of the support structure 603, so that the vent 600 may be removably connected to the second outlet of the device 620 as described previously. It would be appreciated that the connectors 610 may be in other forms, for example, detachable connectors and/or interchangeable connectors. In other embodiments, the vent 600 may be integrally formed with the device 620.

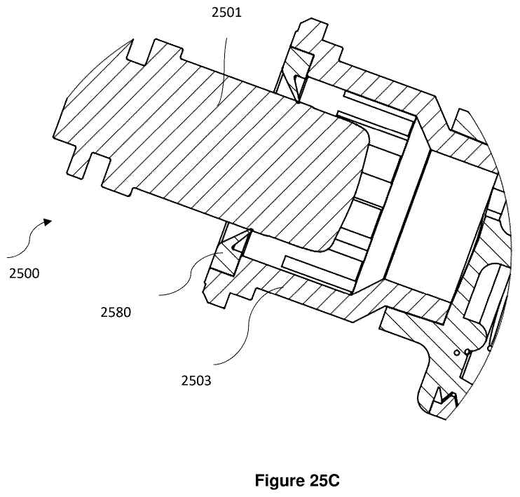

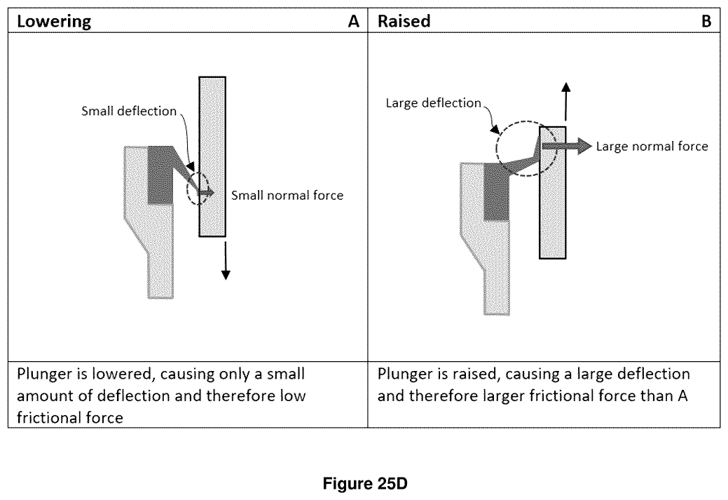

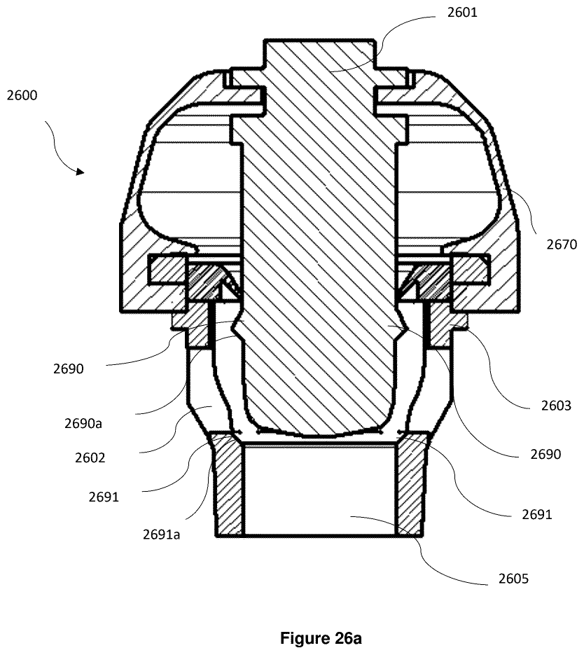

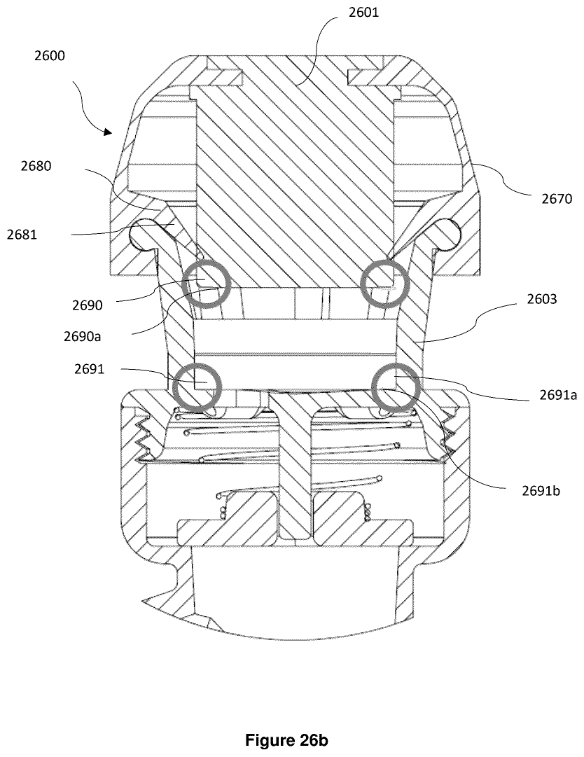

FIGS. 26A and 26B show another exemplary vent 1100 according to the present disclosure.

Similar to previous embodiments, the device 1120 includes an inlet 1124, arranged to receive a flow of breathable gas from the respiratory apparatus 100. A first outlet 1125 of the device 1120 connects to a suitable patient interface (not shown) to convey the breathable gas to the patient. An optional port, for example in the form of a duckbill valve 1123 may be included in the device 1120 for insertion of an auxiliary equipment, such as a catheter for fluid clearance or surfactant delivery to the patient, and/or a monitoring device for monitoring one or more parameters of inhaled and/or exhaled gas, such as a breath indicator device or a gas sampling or detecting device, for example, for detecting CO₂ in the exhaled gas. The device 1120 also includes a second outlet 1126 configured to receive coupling of a vent 1100. The second outlet 1126 may be a PEEP port as described in previous embodiments, or a regular air outlet which does not include a PEEP valve.

The vent 1100 includes an actuator 1101 movable, for example, by an operator to cause a more gentle and gradual transition between different pressures. In this example, the actuator 1101 is formed in a dome shape, including a curved surface 1114, and a substantially flat surface 1113 opposite to the curved surface 1114. The curved surface 1114 may be formed as a semi-spherical shape in some configurations. In at least some embodiments, the actuator 1101 is not tethered or coupled to another component (e.g., a vent cap 1112) of the vent 1100. The actuator 1101 is movable by the operator, for example, by gripping it with two fingers on its left and right side. The vent 1100 includes one or more orifices 1102, arranged to be gradually covered or uncovered by the actuator 1101 as in previous embodiments. In some embodiments, the one or more orifices 1102 may be provided in the vent cap 1112. The vent cap 1112 can be removably coupled to the second outlet 1126 of the device 1120 through suitable connector arrangements. In alternative embodiments, the vent cap 1112 may be integrally formed with the device 1120.

FIGS. 26C and 26D show a plan view and a perspective view, respectively, of an exemplary vent cap 1112 including a plurality of orifices 1102. The vent cap 1112 has been disconnected from the device 1120 for ease of illustration. In this example, when viewed directly from above, the vent cap 1112 is of a generally circular shape as shown in FIG. 26C, with an array of cut outs or openings formed at or near a centre region. The array of openings form the plurality of orifices 1102 of the vent cap 1112 which allow air to flow through, when not covered by the actuator 1101. The plurality of orifices 1102 are divided into four groups in this example, with bridging portions 1103 placed in-between. The plurality of orifices 1102 are configured in a concentric, arch shape, with the openings placed near the centre region of the vent cap 1112 having a shorter length than the openings placed further away from the centre region.

Turning to FIG. 26D, the vent cap 1112 includes a connector 1110 disposed on a lower surface of the vent cap 1112. The connector 1110 includes a plurality of threaded portions. Complimentary threaded portions may be provided in the device 1120 where a coupling between the vent cap 1112 and the device 1120 is to be made. It would be appreciated that the connectors 610 may be in other forms, for example, detachable connectors and/or interchangeable connectors. Alternatively, the vent cap 1112 may be integrally formed with the device 1120. At or near the centre region of the vent cap 1112, a protruding structure 1115 is provided in this example which forms a cross shape in plan view as illustrated in FIG. 26C. The protruding structure 1115 may be used to maintain a minimum distance between the actuator 1101 and the vent cap 1112, to avoid accidental occlusion of the orifices 1102.

In at least some embodiments, the actuator 1101 includes a deformable portion at or near the curved surface 1114. The deformable portion elastically deforms when a force is applied to it, and returns to its shape when the force is removed. The deformable portion could include materials such as silicon, foam, rubber, or similar thereof. Such deformable portion allows the actuator 1101 to flatten its curved surface 1104 as it is pressed against the vent cap 1112, thereby blocking the orifices 1102 of the vent cap 1112. Due to the dome shape of the actuator 1101, orifices closer to the centre region of the vent cap 1112 will be blocked by the actuator 1101 initially if, for example, the force is substantially applied centrally or evenly onto the actuator 1101. As the actuator 1101 is pressed against the vent cap 1102 further, more orifices of the vent cap 1102 will be blocked.

In some embodiments, in addition or as an alternative to providing the deformable portion at or near the curved surface 1114 of the actuator 1101 where it engages the orifices 1102, the vent cap 1112 may also at least in part be formed from an elastically deformable material. In one example, as the actuator 1101 is pressed against the vent cap 1112, the centre region of the vent cap 1112 starts to elastically deform to a shape which is similar to the shape of the curved surface 1114 of the actuator 1101. As the actuator 1101 is moved away from the vent cap 1112, it returns to its original shape.

As the orifices 1102 are blocked, there will be a decrease in the area of the vent 1100 available for the gas from within the respiratory system 1 to exit to ambient air via the second outlet 1126 and the orifices 1102. This elevates the pressure of the breathable gas delivered to the patient, for a given flow rate from the respiratory therapy apparatus 100. As the actuator 1101 is moved away from the vent cap 1112, the orifices 1102 become unblocked, resulting in an increase of the available area of the vent 1100 for the gas to exit from the respiratory system, which in turn lowers the pressure of the breathable gas delivered to the patient, for a given flow rate from the respiratory therapy apparatus 100.

In some embodiments, when the orifices 1102 are fully or substantially blocked, the pressure of the breathable gas delivered to the patient corresponds to PIP, or peak inspiratory pressure. When the orifices 1102 are fully or substantially unoccluded, the pressure of the breathable gas delivered to the patient corresponds to PEEP, or positive end expiratory pressure. When the plurality of orifices 1102 are partially occluded or unoccluded, pressures between PEEP and PIP are delivered to the patient.

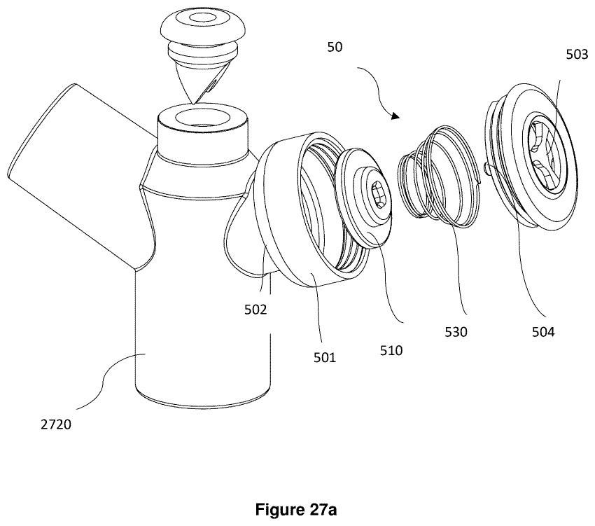

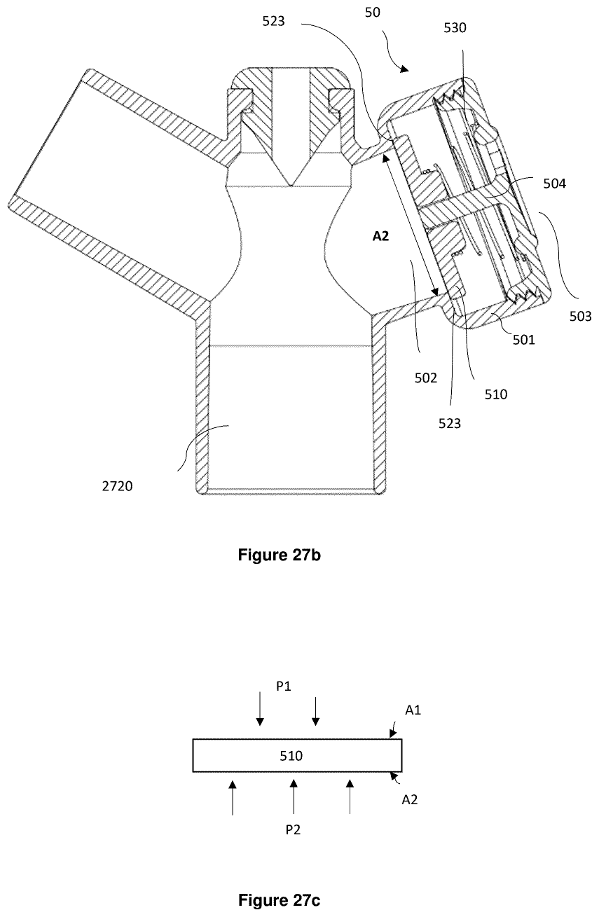

FIG. 27 shows another exemplary vent cap 1112 including a plurality of orifices 1105. The orifices 1105 are formed by an array of concentric, elliptical shaped cut outs which are positioned at or near a centre region of the vent cap 1112.

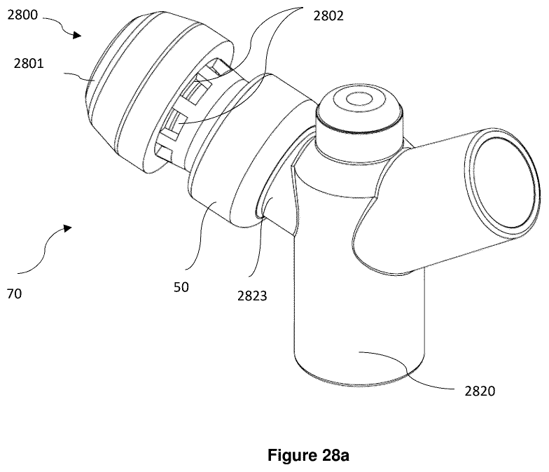

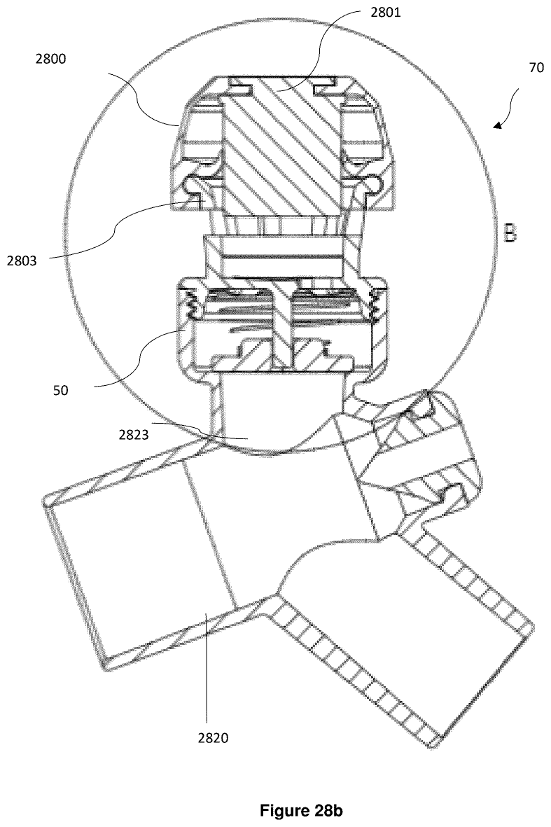

In certain embodiments, a single orifice 1102 may be provided in the vent cap 1112. As the actuator 1101 is pressed against the vent cap 1112, a centre region of the single orifice 1102 will be blocked by actuator 1101 initially, and the blocked region expands from the centre region to an entire area of the orifice 1102, as illustrated by the series of diagrams in FIG. 28. The dark coloured areas indicate area of the single orifice 1102 blocked by the actuator 1101.

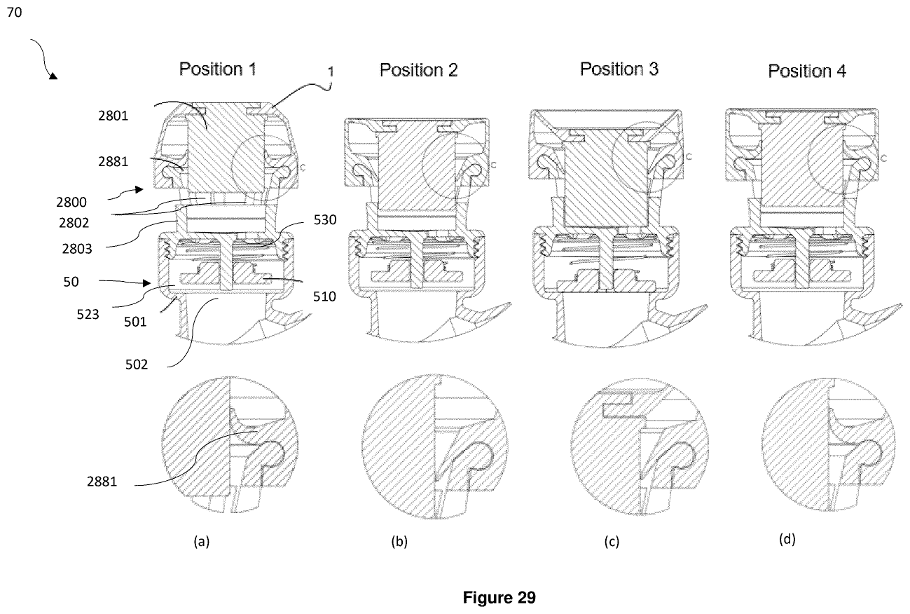

In some embodiments, it may be desirable to include a support structure 1103 in the vent 1110, for holding the actuator 1101 in close vicinity of the vent cap 1102, and/or for avoiding dropping the actuator 1101 by accident. FIGS. 29 and 30 show two examples of such support structures 1103.

In both embodiments, the support structure 1103 includes a body 1105, forming a cavity 1105A, wherein the vent cap 1112 is positioned inside the cavity. The support structure 1103 includes two or more elongate members 1104A, 1104B, 1104C, 1104D which extend in a generally upward direction from the body 1105. A shoulder portion 1106 is formed in the elongate members 1104A, 1104B, 1104C, 1104D and extend inwardly toward the centre of the vent cap 1112. The shoulder portion 1106 of the elongate members 1104A, 1104B, 1104C, 1104D assists to hold the actuator 1101 in close vicinity of the vent cap 1112 without restricting its movement.

In the embodiment shown in FIG. 30, the support structure 1103 includes a circular ring 1107 connecting the shoulder portions 1106 of the elongate members 1104A-D. If a dome shaped actuator 1101 as shown in FIGS. 26A and 26B are used, it will be placed between the elongate members 1104A-D, with the curved surface 1114 facing towards the vent cap 1112, and peripheral regions of the flat surface 1113 will engage the shoulder portions 1106. The vent including such support structures 1103 will still operate in the same manner (i.e. by pressing the actuator against the vent cap 1112) as described previously, however, as the actuator is retained in position by the support structure 1103, the operator can simply operate the device with one finger instead of using two fingers to grip.

FIGS. 31 and 32 show another exemplary vent 1500 with a support structure similar to the support structure of FIG. 30, but with a different actuator configuration. More specifically, the actuator 1501 is configured as a piston. In the example shown, the piston has a dome shaped lower end. FIG. 32 illustrates a side view of the vent 1500 with the support structure of FIG. 30 (i.e., 1503 in FIG. 32 with elongate members 1504) while FIG. 31 illustrates a cross-sectional view (from section A-A) of the vent 1500 of FIG. 32. For ease of illustration, the support structure is omitted in the cross-sectional view in FIG. 31. Different from the embodiments shown in FIGS. 26A and 26B, the dome shaped lower end is formed with a hollow interior. Since less material is being deformed, the force required to deform the dome shaped lower end is considerably less. The actuator 1501 is provided with an engaging member 1505 at an upper surface, over which the operator could place their finger to press down the piston towards the orifice(s).

A relief hole 1540 can be placed at the lower end of the piston, to manipulate the resistance to ensure user comfort and to control the time it takes to occlude the orifices. The relief hole 1540 can provide resistance to compression due to venting out of the relief hole 1540. In this example, connectors 1510 for coupling the vent 1500 to the device (e.g., 1120, 620, 320) are also shown as a plurality of threaded portions. It would be appreciated that the connectors 1510 may be in other forms, for example, detachable connectors and/or interchangeable connectors. Alternatively, the bottom end portions of the vent 1500 may be integrally formed with the device (e.g., 1120, 620, 320) without any connector portions.

FIGS. 33A-C and 34A-C show another exemplary vent 1600 according to the present disclosure.

In this embodiment, the vent 1600 includes an actuator 1601, and a housing 1603. FIGS. 33B and 33C illustrate the actuator 1601 and the housing 1603, respectively, in an disassembled state. FIG. 33A illustrates the actuator 1601 and the housing 1603 in an assembled state, with a perspective cross-sectional view for ease of illustration. The housing 1603 is formed as a hollow enclosure, with a first opening 1604 and a second opening 1606 each disposed at two opposite ends. The first opening 1604 is fluidly connectable to the respiratory system 1, for example, by connecting to an outlet of the device, and the second opening 1606 is configured to movably receive the actuator 1601. Connector portions 1610 are formed at a bottom end of the housing 1603, allowing a coupling to be made between the vent 1600 and the device (e.g., 1120, 620, 320). Alternatively, the housing 1603 may be integrally formed with the device (e.g., 1120, 620, 320) without requiring any connector portions.

In this embodiment, the actuator 1601 includes a hollow body 1605. The actuator 1601 includes a plurality of orifices 1602 disposed along the length of the hollow body 1605. The hollow body may also be provided with one or more air inlets 1650 at a lower end, allowing gas within the enclosure of the housing 1603 to enter into an interior cavity of the actuator 1601. In use, the actuator 1601 is movable between a lifted position and an inserted position with respect to the housing 1603. FIG. 34A shows the actuator 1601 in a substantially or fully lifted position. FIG. 34B shows the actuator 1601 in a partially lifted or partially inserted position. FIG. 34C shows the actuator 1601 in a substantially or fully inserted position. In the substantially or fully lifted position, all of the orifices 1602 are exposed to ambient air, whereas in the substantially or fully inserted position, all of the orifices 1602 will sit within the enclosure formed by the housing 1603, preventing the gas to escape to ambient air via the vent 1600. Between the substantially or fully inserted and the substantially or fully lifted position, one or more of the plurality of orifices 1602 are exposed to ambient air, allowing the gas to exit to ambient air via the vent 1600. Accordingly, the relative position of the actuator 1601 with respect to the housing 1603 at least in part determines the area of the vent 1600 available for the gas the exit from the respiratory system 1 to ambient air, and therefore the pressure of the breathable gas delivered to the patient.

As illustrated in FIGS. 13A-C and 34A-C, the plurality of orifices 1602 are configured to have varying shapes and/or configurations. Some orifices are larger than others. More specifically, the orifice located closer to the lower end of the actuator 1601 has a larger size compared to all the remaining orifices 1602. This is because this is the initial orifice that will be lowered into the enclosure as the actuator 1601 is depressed. This orifice is sized to ensure that when it is lowered into the enclosure, there will be an increase in pressure. If this orifice is not sufficiently large, or not present, there may be minimal pressure rise when gradually lowering the smaller orifices into the enclosure, at least until a few of those orifices have been lowered into the enclosure. This is because the area of the vent available for gas to go through will not be sufficiently increased after covering the smaller orifices and the air will escape through the remaining open orifices without a noticeable increase in pressure. By covering a larger orifice first, the resistance to flow may be large enough for an initial pressure rise, after which covering the smaller orifices will result in a gradual pressure rise. It will be appreciated that the plurality of orifices 1602 may alternatively be configured to have the same shapes and/or size.

When the actuator 1601 is in the fully or substantially lifted position, the pressure of the breathable gas delivered to the patient corresponds to PEEP. When the actuator is in the substantially or fully inserted position, the pressure of the breathable gas delivered to the patient corresponds to PIP. As the actuator is moved between these two positions, pressures between PEEP and PIP are delivered to the patient. FIG. 35 shows an example of a pressure waveform generated by this embodiment. It can be seen that PEEP is provided to the patient for about 0.75 seconds while the actuator is in the fully or substantially lifted position (position 1). As the actuator 1601 is pressed into the housing 1103 (e.g., from position 2 to position 6), pressure starts to increase gradually, until it reaches PIP pressure level when the actuator is in the fully or substantially inserted position (position 6). The rate of change of pressure depends on the speed at which the operator is delivering breaths to the patient, as well as how fast the operator is moving the actuator 1601.

In at least some embodiments, the actuator 1601 is manually operated by the operator to move between the fully or substantially lifted position and the fully or substantially inserted position. The speed of movement is predominantly dependent on the operator. For example, a biasing member 1670 may be provided in the vent 1600, as illustrated in FIGS. 36A-C. The biasing member 1670 maintains the actuator 1601 in the fully or substantially lifted position when no force is applied by the operator as illustrated in FIG. 36A. Similarly, FIG. 36B shows the actuator 1601 in a partially lifted or partially inserted position while FIG. 36C shows the actuator 1601 in a fully or substantially inserted position. In addition, the biasing member 1670 also creates some resistance as the actuator 1601 is depressed, this will provide haptic feedback to the operator and/or help the operator to adjust the speed at which the actuator is moved from the lifted position to the inserted position.

The biasing member may be a spring disposed on the outside of the actuator 1601. In the example shown in FIGS. 36A-C, the biasing member 1670 surrounds the body 1605 of the actuator 1601. The actuator 1601 includes a shoulder 1680, which at least partially extends around a circumference of the actuator 1601. The shoulder 1680 may be formed as a flange as shown in FIGS. 36A-C, or in other suitable configurations. In addition, the housing 1603 includes a recess, for example, a countersunk hollow 1690. The biasing member is held in place by the shoulder 1680 of the actuator 1601 and the countersunk hollow 1690 of the housing 1603.

In some embodiments, a sealing member 1695, such as an O-ring, is also provided to help create a seal when the actuator 1601 is in the fully or substantially lifted position.

FIGS. 37-39 show yet another exemplary vent 2000 according to the present disclosure. This vent 2000 uses similar principles as the exemplary vent 1600, in that an operator needs to depress or insert an actuator 2001 in order to restrict an area of the vent 2000 available for gas to exit from the respiratory system to ambient air, and retract or lift the actuator 2001 to increase the area of the vent 2000 available for gas to escape to ambient air via the vent 2000. FIG. 37 illustrates side, side cross-sectional, and top cross-sectional views (FIGS. 37A, 37B and 37C, respectively) of the vent 2000 with the actuator 2001 in a fully or substantially lifted position. FIG. 38 illustrates similar views of the vent 2000 with the actuator 2001 in a partially lifted or partially inserted position. FIG. 39 illustrates similar views of the vent 2000 with the actuator 2001 in a fully or substantially inserted position. The vent 2000 includes a housing 2003, configured to receive the actuator 2001 as it is pressed down. The housing 2003 has two openings, one at each end. A first opening 2005 is configured to be blocked by the actuator 2001 as it is lowered into the housing 2003. A second opening 2007 is configured to movably receive the actuator 2001.

When the actuator 2001 is in a fully or substantially lifted position, gas from within the respiratory system 1 escapes from the vent 2000 via an area between the actuator 2001, and an interior wall of the housing 2003. As the actuator 2001 is gradually depressed, this area decreases in size, hence restricting air flows and elevates the pressure delivered to the patient. To achieve a gradual change of the area between the actuator 2001 and the housing 2003, the actuator 2001 and/or the housing 2003 may be configured to have a tapered profile as illustrated in FIGS. 37-39. For example, the actuator 2001 includes a body portion 2011, including a first end 2013, which the operator may engage, and a tapering end 2015 opposite to the first end 2013. A diameter of the body portion 2011 decreases towards the tapering end 2015, to achieve a gradual reduction of the area between the actuator 2001 and the interior wall of the housing 2003 as it is depressed into the housing 2003. The tapering end 2015 may also assist a smooth insertion into the first opening of the housing 2003.

In another embodiment, the actuator 2001 may be configured in a substantially cylindrical form without having a tapering end. Instead, the housing 2003 is formed with tapered walls, such that a diameter of the housing 2003 reduces towards the first opening 2005 where the actuator 2001 inserts into. This will also achieve a gradual reduction of the area between the actuator 2001 and the wall of the housing 2003 as the actuator 2001 lowers into the first opening 2005.

When the actuator 2001 is fully or substantially inserted such that the tapering end 2015 extends fully or substantially into the first opening 2005 of the housing 2003 as illustrated in FIG. 39, the area between the actuator 2001 and the housing 2003 is completely closed off as illustrated in FIG. 39(C), and air flow through the vent 2000 is also stopped.

In at least some embodiments, one or more additional orifices may be formed in the wall of the housing 2003 to allow the gas to flow through when exiting via the vent 2000. In earlier embodiments, the one or more orifices are arranged to be occluded or unoccluded by the actuator as it is moved. In this embodiment, the one or more orifices within the wall of the housing 2003 are not configured to be occluded or unoccluded to cause a pressure change. Instead, they are provided as additional air outlets which allow the gas to escape from the vent 2000 more easily. The one or more orifices are designed to ensure that they have no significant impact on the restriction of air flow. In some embodiments, the one or more orifices are greater than the area between the actuator 2001 and the housing 2003 at any time during operation.

In some embodiments, the vent 2000 could also include a biasing member (not shown) which maintains the actuator 2001 in the fully or substantially lifted position, and creates some resistance when the actuator 2001 is depressed, for example, by the operator. FIG. 40 shows a cross-sectional diagram of an exemplary vent 2100 configured to include such biasing member. Similar to the exemplary vent 2000, the vent 2100 includes an actuator 2101 movable between a fully or substantially lifted position and a fully or substantially inserted position, to change an area of the vent 2100 available for gas within the respiratory system to exit via the vent 2100. More specifically, in the fully or substantially inserted position, a lower end 2105 of the actuator 2101 is inserted into an opening of a housing 2103, and completely blocks the air flow path via the vent 2100. In a partially lifted or partially inserted or a fully/substantially lifted position, a gap is created between the actuator 2101 and an interior wall of the housing 2103, which forms the area of the vent 2100 available for gas to flow through in order to exit the respiratory system 1.

In this example, the actuator 2101 is provided with a shoulder 2106 towards an upper end 2013 thereof. The shoulder 2106, which may be formed as a flange, may partially or fully extend around a circumference of the actuator 2101. A substantially flat supporting surface 2103a surrounds an upper opening of the housing 2103 where it movably receives the actuator 2101. The biasing member (not shown) may be supported in place by the shoulder 2106 of the actuator 2101, and the flat supporting surface 2103a of the housing. In some embodiments, the biasing member may be a spring and the actuator 2101 is positioned in a hollow centre of the spring, similar to the exemplary actuator 1601 shown in FIGS. 36A-C.

As the actuator 2101 is pressed down, the spring becomes compressed, which at least in part provides some control over the speed of moving the actuator 2101. As the operator releases their finger from the actuator 2101, the spring biases the actuator 2101 towards the fully or substantially lifted position. The spring determines how the actuator 2001 moves, and, therefore, the profile of the tapering end of the actuator 2001 and the spring (spring constant) are selected/configured relative to each other to achieve the desired effect. A spring can be selected for a given profile of tapering end of the actuator 2001; and/or a profile of the tapering end of the actuator 2001 can be designed/selected for given spring parameters/range of parameters, e.g., spring constant. A significant effect it has may be on the total length of the tapering end of the actuator 2001. If the spring can be compressed by length L, then the tapering end may be less than L, in order to undergo a full range of motion before the spring ‘bottoms out’.

The housing 2103 may also include one or more recesses 2104 which are formed in the upper opening of the housing. The one or more recesses 2104 may take the form of, for example, one or more threaded portions. One or more protrusions 2105 may also be formed on the body of the actuator 2101. The one or more protrusions may take the form of, for example, one or more threaded portions configured to correspond with the threaded portions (i.e., 2104) in the upper opening of the housing 2103. The recess(es) 2104 and protrusions(s) 2105 cooperate to set a maximum lifted position for the actuator 2101, so it does not become accidentally disengaged from the housing 2103.

Connector portions 2110 may be provided in a lower end of the housing 2103 in this example, allowing a removable connection to be made between the vent 2100 and a suitable component of the respiratory system 1, such as air outlet of the device (e.g., 1120, 620, 320) which fluidly connects a conduit assembly and a patient interface. The air outlet may be a PEEP port of a T-piece device in some configurations. It should be appreciated that the connector portions 2110 are optional and are provided as an example, other forms of releasable connectors may also be used. In addition, in some embodiments it may be desirable to have the vent 2100 integrally formed in an outlet of the device (e.g., 1120, 620, 320) which means the connector portions 2110 may not be required.

FIG. 41 shows an example of a pressure waveform of the breathable gas delivered to the patient when the vent 2000 of FIGS. 37-39 is used. Here, PEEP is delivered from 0 to 0.75 seconds while the actuator 2001 is in the fully or substantially lifted position (i.e., position 1). As the actuator 2001 is pushed down, the area between the actuator 2001 and the housing 2003 is gradually closed. PIP is delivered while the actuator 2001 is in the fully or substantially inserted position (i.e., position 5). This offers a controlled rise in pressure from PEEP to PIP. Additionally, as the actuator 2001 lifts, the area between the actuator 2001 and the housing 2003 is gradually opened. This offers a controlled drop in pressure from PIP to PEEP.

In some embodiments, a guiding member may be provided in the vent 2001, which helps to maintain the actuator 2001 in an upright direction as it moves between lifted and inserted positions. FIG. 41 shows an example of such guiding member 2096, formed as a vertical rod and positioned below the tapering end 2015 of the actuator 2001. In FIG. 41 like components and features to those described with reference to FIGS. 37-39 are shown with like reference numerals. A receiving channel 2097 is formed in the actuator 2001, and receives the guiding member 2096 as it is slowly pressed down.

Similar to previous embodiments, connector portions 2010 are formed in a base of the vent 2000, allowing a coupling to be made between the vent 2000 and a suitable component of the respiratory system 1, such as an outlet of a device which fluidly connects between a conduit assembly and a patient interface. In some embodiments, the outlet may be a PEEP port of the device (e.g., 1120, 620, 320). The base also acts as a seat for the housing 2003 of the vent 2000, and accommodates a lower end of the housing 2003. In addition, with reference to FIG. 42 which shows a plan view of the base, one or more arms 2098 may be provided in the base to support the guiding member 2096 in a center of the base. The guiding member 2096 may be positioned to extend in a generally vertical direction and aligns with the receiving channel 2097 of the actuator 2001.

FIGS. 43A-D and 44A-D illustrate yet another embodiment of a vent 2200. FIGS. 43A-D illustrate side cross-sectional views of the vent 2200 with various positions of an actuator, and FIG. 44A-D illustrates corresponding side on views of the vent 2200.

Similar to the exemplary vent 2000, the vent 2200 includes a housing 2203, comprising a first and a second opening at opposite ends of the housing 2203. The first opening may be fluidly connected to an air outlet of the respiratory system 1 when delivering respiratory therapy, and the second opening is configured to movably receive an actuator 2201. The housing 2203 includes a body extending between the first and the second opening, which forms a hollow cavity within the housing 2203. A side wall of the body tapers from the second opening to the first opening, such that the first opening is of a smaller diameter than the second opening. A plurality of orifices 2202 are formed in the side wall of the body, as shown in FIGS. 44A-D, configured to allow gas from within the respiratory system 1 to flow through depending on the relative position between the actuator 2201 and the housing 2203.

The actuator 2201 is arranged to move between a fully or substantially lifted position (as for example shown in FIG. 43D) and a fully or substantially inserted position (as for example shown in FIG. 43A) to adjust the area of the vent 2200 available for gas to flow through when exiting from the respiratory system 1, which in turn adjusts the pressure of the breathable gas delivered to the patient. In the fully or substantially lifted position, the gas from within the respiratory system 1 can flow through the first opening of the housing 2203, and then through the orifices 2202, to exit from the respiratory system 1 to ambient air. This has an effect on lowering the pressure delivered to the patient as compared when the actuator 2201 is in the partially inserted, partially lifted or fully/substantially inserted position. In the fully or substantially inserted position, the actuator 2201 is lowered into the housing 2203, to occlude the first opening of the housing 2203. This will block the air flow path, such that air cannot escape the respiratory system 1 via the vent 2200, which elevates the pressure delivered to the patient. When the actuator 2201 is in the fully or substantially lifted position, the pressure of the breathable gas delivered to the patient corresponds to PEEP, and when the actuator 2201 is in the fully or substantially inserted position, the pressure of the breathable gas delivered to the patient corresponds to PIP. As the actuator 2201 is moved between these positions, pressures between PEEP and PIP are delivered to the patient.

The vent 2201 may include a deformable membrane 2270 which assists with the movement of the actuator 2201. As shown in FIGS. 43A-D, the membrane 2270 forms a chamber extending between the second opening of the housing 2203, and a shoulder of the actuator 2201. The membrane 2270 is configured such that it biases the actuator 2201 in the lifted position when no force is applied to the actuator 2201. As a pressing force is applied to the actuator 2201, for example, by an operator, the membrane 2270 starts to deform. As the actuator 2201 moves past a deflection point of the membrane 2270, it biases the actuator 2201 into its fully or substantially inserted position.

The mechanism that allows the membrane 2270 to bias the actuator 2201 in its fully or substantially lifted position and into its fully or substantially inserted position, is controlled by elasticity and geometry of the material used to construct the membrane 2270. FIGS. 45A-C and 46A-C show more details as to how the mechanism works. In the example shown, the membrane 2270 includes a first member 2270-1 and a second member 2270-2, which are joined at an angle. The joint between the two members functions as a flexible hinge, allowing the relative flexing movement of the two members of the membrane 2270. In FIG. 45A, the actuator 2201 is in the fully or substantially lifted position and members 2270-1 and 2270-2 are both at rest as shown in FIG. 46A. This is a stable and resting position for the membrane 2270. FIG. 46A also shows an equivalent spring at rest for illustration purposes.

As the actuator 2201 is pressed down, the membrane 2270 starts to stretch or deform, until it reaches a deflection point as indicated in FIG. 45B and FIG. 46B. At the deflection point, the second member 2270-2 may be substantially horizontal, causing the first member 2270-1 to deflect. FIG. 46B also shows an equivalent spring with maximum force for illustration purposes. Once the actuator 2201 moves past this deflection point, due to the elasticity of the membrane, it biases the actuator 2201 into the fully or substantially inserted position, as indicated in FIG. 45C. As shown in FIG. 46C, the second member 2270-2 is below the horizontal position, meaning that the first member 2270-1 is less deflected compared to the state as shown in FIG. 46B. This is a semi-stable position for the membrane 2270, which is more stable than the state shown in FIG. 46B but less stable than the state shown in FIG. 46A. As soon as the force applied to the actuator 2201 is removed, the membrane 2270 moves itself back to the stable position of FIG. 46A and FIG. 45A.

The actuator 2201 shown in FIGS. 43A-D and 44A-D is similar to the actuator 2001 shown in FIGS. 37-39 and 41. It may include a body portion 2211, including a first end 2213 and a tapering end 2215. A diameter of the body portion 2211 decreases towards the tapering end 2215, to cause a gradual reduction of the area between the actuator 2201 and the housing 2203 as it moves from the fully or substantially lifted position to the fully or substantially inserted position. A guiding member 2296 may also be provided in the vent 2201, which helps to maintain the actuator 2201 in an upright direction as it moves between fully or substantially lifted and inserted positions. FIGS. 43A-D and 44A-D shows an example of such guiding member 2296, formed as a vertical rod and positioned below the tapering end 2215 of the actuator 2201. A corresponding receiving channel 2297 is formed in the actuator 2201, and receives the guiding member 2296 as the actuator 2201 moves into its fully or substantially inserted position.

Similar to previous embodiments, connector portions 2210 may be formed in a base of the vent 2200, allowing a coupling to be made between the vent 2200 and a suitable component of the respiratory system 1, such as an air outlet of a device which interconnects a conduit assembly and a patient interface. The air outlet may be a PEEP port of the T-piece device (e.g., 1120, 620, 320) as in previous embodiments.

In at least some embodiments, the actuator 2001, 2101 or 2201 may be formed as a straight plunger without necessarily having a tapering end, that is, a diameter of the actuator could remain substantially constant along the length of the actuator. The configuration of the housing could be adjusted accordingly to accommodate the actuator as it is inserted into the housing.

FIGS. 47A and 47B show a further embodiment of a vent 2500, coupled to a device 2520 of the respiratory system 1. The intention of this embodiment is to provide an asymmetric waveform profile where pressure transitions between PIP and PEEP occur in different time durations. More specifically, the embodiment aims to provide a longer duration when PIP transitions to PEEP, and a shorter duration when PEEP transitions to PIP. The benefit of prolonging the transition from PIP to PEEP, is to make the pressure change more gradual and further reduce any potential injury caused by rapid pressure changes. Or alternatively, the embodiment may be configured such that it provides a shorter duration when PIP transitions to PEEP, and a longer duration when PEEP transitions to PIP. This embodiment may be combined with some of the previous described embodiments (for example, the embodiments illustrated in FIGS. 43A-D, 44A-D and 45A-D) such that an asymmetric pressure waveform is created.

As mentioned previously, the pressure waveform generated by various embodiments of the vent is dependent on how fast the actuator is moved by an operator, and the desired therapy to be provided to the patient. The present embodiment deliberately introduces some resistance to the actuator as it is moved in a predetermined direction. When the actuator is moved in a different direction, the resistance exerted onto the actuator is reduced. This may be achieved, for example, by introducing a member into a movement path of the actuator, and creating a level of friction between the member and the actuator when it is moved in the predetermined direction.

In one form, the member could be a length of material which extends into the movement path of the actuator. FIG. 47A illustrates an example of such member 2580, configured to slow down the movement of the actuator 2501 as it moves from a fully or substantially inserted portion back to a fully or substantially lifted position. This may be preferred when a sharper increase of inspiratory pressure and a more gentle collapsing of the patient's lungs during the expiratory phase are preferred. A close up view of the member 2580 is provided in FIG. 47B. FIGS. 48A and 48B show further details of how the member 2580 deflects to apply the friction force to the actuator 2501, as the actuator 2501 moves in different directions.

The member 2580 is disposed on an internal surface of the housing 2503, and more specifically, at an opening of the housing 2503 where it movably receives the actuator 2501. The member 2580 may extend partially around the opening of the housing, or it may form a ring and extend the circumference of the opening. As illustrated in FIGS. 47A and 47B, the member 2580 includes a support 2582, and a flap 2581, which extends out at an angle Θ with respect to the support 2582. As illustrated, angle Θ may be less than 90°. The support 2582 and the flap 2581 may both be constructed from deformable materials, allowing the flap 2581 to deflect or flex with respect to the support 2582. As the actuator 2501 is pushed down to its fully or substantially inserted position, a lower end and/or side walls of the actuator 2501 contact and flex the flap 2581, such that the flap 2581 moves towards the support 2582. When the actuator 2501 starts to move up to return to its fully or substantially lifted position, due to the orientation of the flap 2581, a greater frictional force is exerted onto the actuator 2501 by the flap 2581, which has an effect in slowing down the movement of the actuator 2501. This helps to create a smooth exhalation profile due to the controlled movement of the actuator 2501, without any operator input or with limited operator input.

FIGS. 48A and 48B illustrate how the flap 2581 deflects as the actuator 2501 is moved in different directions. As shown in FIG. 48A, as the actuator 2501 is pressed or lowered, there is a small amount of deflection of the flap 2581 and therefore low frictional force/resistance to actuator movement. In contrast, as shown in FIG. 48B, as the actuator 2501 is lifted or raised, there is a larger deflection of the flap 2581 and therefore relatively larger frictional force/resistance to actuator movement.

The parameters which impact how much resistance can be provided by the member 2580 include, at least, a length of the flap 2581, the length of the overlap L of the flap 2581 and the actuator 2501 and/or the angle Θ at which it extends out with respect to the support 2582 (as shown in FIG. 47B). These parameters will determine the amount of contact the actuator 2501 has with the flap 2581. The amount of contact then determines how much the actuator 2501 can be slowed down when it is returning to its fully or substantially lifted position. In some embodiments, the angle Θ is within a range of 40° to 70°. In addition, a further factor which influences resistance is the coefficient of friction between the member 2580 and the actuator 2501. Selecting a material for the member 2580 which exhibits a greater coefficient of friction with the actuator 2501 increases the resistance provided.

In the example shown in FIG. 47B, the flap 2581 extends inwardly toward a centre of the housing 2503, and downwardly from an upper edge of the support 2582. This has the effect of slowing down the movement of the actuator 2501 as it returns to its fully or substantially lifted position. In an alternative embodiment, the flap 2581 may be configured such that it extends inwardly toward the centre of the housing 2503, but upwardly from the support 2582. This will create an opposite effect to the example shown in FIG. 47B, and slows down the movement of the actuator 2501 as it is pushed from the fully or substantially lifted portion to the inserted position.

In at least some embodiments, the actuator may comprise a sealing portion to improve the sealing between the actuator and the vent housing, particularly when PIP is administered. In some embodiments, a complimentary sealing portion may be provided in the housing, being configured to engage the sealing portion of the actuator during PIP delivery. Examples of such sealing portions are illustrated in FIGS. 49 and 50.

With reference to FIG. 49, the sealing portion may comprise a protrusion 2690 formed on an exterior surface of actuator 2601. The protrusion 2690 may extend fully, partially, or at least a substantial portion, of the circumference of the actuator 2601. From a side view, the protrusion 2690 may have a triangular cross-sectional profile, formed by two angled surfaces. At or towards the lower end of the housing 2603, a complimentary sealing portion 2691 is formed on an interior wall of the housing 2603, at a location where a seal is to be created between the actuator 2601 and the housing 2603, when the actuator 2601 is lowered into the housing to reach its fully or substantially inserted position. In the embodiment shown in FIG. 49, the complimentary sealing portion is formed as a recess, or a chamfer 2691 in the interior wall of the housing 2603, comprising a surface 2691a that is placed at a similar sloping angle as a lower surface 2690a of the protrusion 2690. As the actuator 2601 is lowered into the inserted position, to block a first opening 2605 of the housing 2603, the protrusion 2690 of the actuator 2601 is arranged to rest on, or engage the complimentary sealing portion 2691 of the housing 2603, thereby improving the seal between the actuator 2601 and the housing 2603.

FIG. 50 shows another example of how such sealing may be achieved between the actuator 2601 and the housing 2603. In this embodiment, the vent 2600 is positioned on top of a PEEP valve (discussed in more detail below), which is fluidly coupled to a PEEP port of a T-piece device (e.g., 1120, 620, 320). The sealing portion 2690 of the actuator 2601 is formed by one or more surfaces of an edge portion of the actuator 2601. For example, the actuator 2601, which is formed as a plunger, is configured to have a cylindrical shaped main body, with a substantially flat bottom surface 2690a. The housing 2603 includes a complimentary sealing portion 2691, formed by an internal vertical side wall 2691a and a horizontal surface 2691b at the bottom of the housing 2603. As the actuator 2601 is depressed, a flat horizontal surface 2690a of the actuator is allowed to rest on the horizontal surface 2691b of the housing, thereby improving the sealing between the actuator 2601 and the housing 2603. It will be appreciated that the sealing arrangements illustrated in FIGS. 26a and 26b are provided as examples only. Other types of sealing portions may be provided as alternatives.

FIG. 50 also illustrates another configuration of a member 2680, which may be used to slow down the movement of the actuator 2601 as it moves in a selected direction, for example, when the actuator 2601 moves from the fully or substantially inserted portion back to the fully or substantially lifted position. The member 2680 is formed as flap 2681, which extends into the movement path of the actuator 2601, and contacts a side wall of the actuator 2601 to apply a frictional force. In this embodiment, the flap 2681 is directly formed as part of the deformable membrane 2670.

### A Pressure Regulating Device

As mentioned above, a PEEP valve may be provided in the PEEP port of a device, which actuates at a selected pressure, to allow the breathable gas to vent externally and regulate the gas pressure administered to the patient. Examples of such PEEP valves are at least shown in international patent application number PCT/NZ2013/000111 (published as WO 2014/003578), and in U.S. provisional patent application 63/366,660. The vent of the present disclosure may be used in conjunction with a PEEP valve, and more preferably, a flow independent PEEP valve, to further improve the mechanical ventilation delivered to patients during resuscitation or other types of respiratory therapy.

In a further embodiment, the present disclosure provides a pressure regulating device, including at least one of the vent described above, and additionally, a flow independent PEEP valve operatively coupled to the vent. By “flow independent” it is meant that the PEEP valve is configured to compensate for unintentional flow variations, such as interface leaks, or auto-PEEP, often experienced by the respiratory system 1 near the patient's end, such that the PEEP pressure delivered to the patient remains within a targeted PEEP pressure range. This combination enables a more gentle delivery of PIP and PEEP through the gradual occlusion of the PEEP port, as described above in relation to various embodiments of the vent. Further, due to the inclusion of the flow independent PEEP valve, the gas pressures delivered to the patient are maintained within a predetermined pressure range when PEEP is administered, independent of flow variations caused by interface leaking or auto-PEEP.

An example of a spring controlled PEEP valve 50 is shown in FIGS. 51 and 52. The valve 50 includes a valve body 501, defining an inlet 502, and an outlet 503, via which a gas flow may enter and exit the valve 50 when the valve 50 is open. A controller 510 is accommodated within the valve body 501, for example in the form of a valve disk as illustrated in FIGS. 51 and 52, configured to move along a shaft 504 to enable opening and closing of the valve 50. The movement of controller 510 at least in part assists with the pressure regulation by the valve 50, particularly during PEEP delivery.

As indicated in FIG. 52, a peripheral region of the controller 510 is configured to engage or rest on a valve seat 523 formed by an internal wall of the body 502. As the valve 50 is fluidly coupled to the PEEP port of the device 2720, a lower surface of the controller 510 is exposed to the gas at the inlet 502 of the valve 50. The controller 510 is subject to a lifting force (F_{lift}), created by a differential gas pressure between an upper and a lower surface of the controller 510, in a direction which is aligned with an axis of the shaft 504. With reference to FIG. 53, the lifting force (F_{lift}) the controller 510 is subject to, equals P₂*A₂−P₁*A₁, wherein P₁ and P₂ are the gas pressures experienced by the upper and lower surfaces of the controller 510 respectively, and A₁, A₂ represent the upper and lower area of the controller 510 which are exposed to the relevant gas pressures. When the valve 50 is configured to vent gases of the respiratory system 1 directly to ambient air, and a difference between A₁ and A₂ is relatively small, or negligible, the equation used to calculate F_{lift} may be simplified to P*A, where P is gas pressure at the inlet 502 of the valve 50 (e.g. 5 cm H₂O), and A is the area of the controller 510 exposed to the gas pressure. Alternatively, when the outlet 503 of the valve 50 is fluidly connected to a vent of the present disclosure, such as shown in FIGS. 54, 55, and 56A-D, and provided that the difference between A₁ and A₂ is still small or negligible, the equation used to calculate F_{lift} may be simplified to ΔP*A, where ΔP equals the pressure differential across the upper and lower surface of the controller 510, and A is the lower surface area of the controller 510 exposed to the pressure differential ΔP.

Turning back to FIG. 52, a preloaded biasing member 530 is sandwiched between the controller 510, and a top wall of the valve body 501. That is, the height of biasing member 530 in FIG. 52 is smaller than an uncompressed natural height of the biasing member 530. As the biasing member 530 is already compressed, it applies a resistance force (F_{bias}) onto the controller 510, which pushes the controller 510 toward the inlet 502 of the valve 50, until it engages the valve seat 523. When in this seated position, the controller 510 closes off a gas flow path between the inlet 502 and the outlet 503, minimising or preventing any gas flow through the valve 50.

In at least one form, the biasing member 530 is a spring. Accordingly, the resistance force created by the biasing member 503 may be calculated from F_{bias}=k*(x_initialCompression+x_lift), where
k=spring constant;
x_initial compression=initial compressed length of the spring, when the flow path is closed off (i.e. a difference between the original uncompressed length of the spring, and the length of the spring after it has been initially compressed and placed within the valve body 501);
x_lift=displacement of the controller 510 from the valve seat 523, at pressure P.

It will be appreciated that the resistance force caused by the biasing member 530 is a variable resistance force, due to x_lift. When the controller 510 is biased against the valve seat 523, x_lift equals zero, as there is no relative displacement of the controller 510 from the valve seat 523 yet. The initial variable resistance force applied by the biasing member 530 may be simplified to F_{bias}=k*x_initialCompression. As the controller 510 is lifted off the valve seat 523, and starts sliding along the shaft 504 with the valve body 501, x_lift equals the displaced distance of the controller 510 with respect to the valve seat 523, which is a variable parameter.

In addition to the variable resistance force (F_{bias}) applied by the biasing member 530, the controller 510 is also subject to an upward lifting force (F_{lift}), as mentioned above. The lifting force is dependent on the pressure differential between the upper and lower surfaces of the controller 510 (ΔP), and the area of the controller 510 exposed to such pressure (F_{lift}=ΔP*A). Accordingly, the position and movement of the controller 510 with respect to the valve seat 523 is determined by the relative strengths of the two forces F_{bias} and F_{lift}. Using the equations mentioned above, a minimum differential pressure that is required to lift the controller 510 off the valve seat 523 can be determined, which is k*x_initialCompression/A. This minimum differential pressure level determines the selected pressure level at which the valve 50 opens, as well as the predetermined pressure range which the valve 50 is configured to regulate. When the flow independent valve 50 is used to regulate PEEP pressures, the spring constant, x_initial compression, and the exposed area A of the controller 510 are selected such that the selected pressure level at which the valve 50 opens, is within the targeted PEEP pressure range.

When the valve 50 is configured to vent gases directly to ambient air, such as shown in FIGS. 51 and 52, the pressure differential across the controller 510 may be considered as the gas pressure at the valve inlet 502. If this pressure is lower than the selected pressure level, F_{lift} is not sufficient to lift the controller 510 off the valve seat 523. Accordingly, the net effect of two forces maintains the controller 510 in its seated position on the valve seat 523 (F_{bias}>F_{lift}). As gas pressure at the inlet starts to increase, it translates into an increased F_{lift}. As the gas pressure exceeds the selected pressure level, which causes F_{lift} to be greater than F_{bias}, the lifting force will overcome the variable resistance force applied by the biasing member 530 (F_{lift}>F_{bias}), and lifts the controller 510 above valve seat 523.

As the controller 510 is lifted off the valve seat 523, a gap is formed between the edge of the controller 510, and the valve seat 523. The gas flow may then start to enter the valve body 501 through this gap, and flows out of the valve 50 via the outlet 503. In other words, the gas flow path between the inlet 502 and the outlet 503 of the valve body 501 is now open, and the gas within the respiratory system 1 can now flow through the valve 50. If the gas pressure is higher than the selected pressure level, even though the controller 510 has already been lifted off the valve seat 523, F_{lift} will continue to displace the actuator 510 along the shaft 504, to increase the gap between the controller 510 and the valve seat 523, allowing more air to flow through the valve 50. At the same time, the lifting of the controller 510 causes the biasing member 530 to compress further, which increases the variable resistance force generated by the biasing member 530, until it reaches an equilibrium position where F_{lift} equals F_{bias}, at which point the controller 510 is not displaced any further away from the valve seat 523. The controller 510 remains in that position to vent the gas flow externally, until the gas pressure changes again. If F_{lift} is smaller than F_{bias}, the net effect of the two forces will start moving the controller 510 towards the valve seat 523, reducing the size of the flow path within the valve 50.

In addition to selecting a suitable spring constant (k), initial compression of the spring (x_initialcompression), and the exposed area of the controller 510 (A), there are other design considerations which contribute to how the valve 50 may be configured, at least some of which are set out below.

Gas flow rate via the valve 50: to regulate PEEP pressure, the gas flow rate that the valve 50 is able to regulate may be within a range of 0 to 20 L/min when used with infants. This range is approximately the same as the source flow rate which the respiratory system 1 is set to provide to a patient.

Predetermined range of PEEP pressure: for infant resuscitation, the targeted PEEP pressure is usually within a range of 4 to 15 cm H₂O. The valve 50 may be configured such that it is able to regulate the gas pressure within the respiratory system 1, such that it stays within this range. In some embodiments, the valve 50 is configured to regulate the pressure of the breathable gas within the respiratory system 1 by a variation of −2 to +2 cm H₂O, −1 to +1 cm H₂O, or by a variation of −0.5 to +0.5 cm H₂O. That is, if the respiratory system 1 is set to deliver PEEP to a patient at 5 cm H₂O, at a given or predetermined flow rate, then the valve 50 is configured to regulate the PEEP pressure such that it stays within a range of 3-7 cm H₂O, or 4-6 cm H₂O, or 4.5-5.5 cm H₂O, at that given flow rate, regardless of any unintentional flow variations experienced by the system.

Depth of the valve: the depth of the valve 50 at least in part determines the initial compressed length of the biasing member 530 (x_initialcompression), and a maximum x_lift that is able to be achieved. In at least one embodiment, the depth of the valve is approximately 3 to 4 mm.

Configuration of the biasing member 530: configuration of the biasing member includes selection of suitable spring constant, spring wire diameter, sizes of the spring coils, number of the spring coils, spring pitch, and so on. In at least one embodiment, the spring constant is less than 0.05 N/mm. Preferably, the spring constant is within a range of 0.005 to 0.02 N/mm.

Controller: The exposed area of the controller 510 and the dimension of the valve inlet 502 may be selected so that the pressure to open the valve 50, and the pressure that the valve regulates are similar. Further, the exposed lower surface area of the controller 510, and the spring constant, are selected such that a relatively small displacement of the controller 510 (i.e. in mm range, or a fraction of a millimetre) with respect to the valve seat 523 is sufficient to allow the valve 50 to achieve its pressure regulation effect. The cross-sectional area of the controller is preferably smaller, or considerably smaller than an inner transverse dimension of the valve body 501, so there is no significant additional resistance to flow when gases pass between an internal side wall of the valve body 501 and the controller 510. In at least some embodiments, the cross-sectional area of the controller 510 is between 50-320 mm2.

An exemplary pressure regulating device is described below with reference to FIGS. 54, 55 and 56A-D. The pressure regulating device 70 includes a vent 2800, which has similar or identical configuration as the vent 2600 illustrated in FIG. 50. It will be appreciated that the vent 2800 may be replaced with any one of the vents described above. Similarly, the illustrated embodiment includes a flow independent PEEP valve 50 which is controlled by a spring, as for example described in U.S. provisional application 63/366,660. This valve 50 may alternatively be replaced with another suitable flow independent PEEP valve, for example, an umbrella valve as described in international patent application number PCT/NZ2013/000111 (published as WO 2014/003578).

FIG. 54 shows a side perspective view of a T-piece device 2820, coupled to a pressure regulating device 70 at its PEEP port 2823. FIG. 55 shows a side cross-sectional view of the pressure regulating device 70. The pressure regulating device 70 in this example includes the vent 2800, and PEEP valve 50 such as illustrated in FIG. 52. The vent 2800 and the PEEP valve 50 may be integrally or detachably connected together, for example, through suitable connector portions. In one embodiment, complementary connector portions may be formed in the housing 2803 of the vent 2800, and in the body 501 of the PEEP valve 50, allowing the two components to be detachably connected to each other. In use, when the PEEP valve 50 and the vent 2800 are both open, the breathable gas may flow through both components and vent externally to the ambient air.

FIGS. 56A-D illustrate how the pressure regulating device 70 operates as it transitions between PEEP and PIP. The illustrated embodiment includes a flap 2881 configured to slow down the movement of the actuator 2801 as it returns from the fully or substantially inserted position to the fully or substantially lifted position. It will be appreciated that the flap 2881 may be omitted in other embodiments of the pressure regulating device 70.

FIG. 56A indicates where the actuator 2801 of the vent 2800 and the controller 510 may likely be positioned (position 1), when PEEP is delivered to the patient. If the pressure differential across the controller 510 exceeds the selected pressure level at which the valve 50 is configured to open, the controller 510 is lifted off the valve seat 523, opening the valve 50. As no external force is applied onto the actuator 2801 during PEEP delivery, the actuator 2801 is also in its raised position, and preferably at its maximum height with respect to the housing 2803, allowing the breathable gas to exit the vent via one or more orifices 2802 formed on the wall of the housing 2803. When the actuator 2801 and the controller 510 are both lifted, the entire gas flow path within the pressure regulating device 70 is open, allowing gas flow through the device 70 freely.

Flow variations, such as auto-PEEP or interface leaks, often occur near the patient's end. The PEEP valve 50 compensates for such flow variations, by varying the relative displacement of the controller 510 with respect to the valve seat 523. For example, in the event of auto-PEEP, the pressure and/or flow at the valve inlet 502 may be elevated to a higher level due to patient's breathing. The controller 510 is displaced further away from the valve seat 523, allowing more gas to enter the valve 50 and flow through the pressure regulating device 70. Alternatively, if there is an interface leak, the gas pressure at the valve inlet may be lower. The lower gas flow will create a lower pressure, which will cause the controller 510 to move closer to the valve seat 523, thereby increasing restriction, reducing the amount of breathable gas entering the valve 50 and venting externally. In this way, the PEEP valve 50 assists with regulating the gas pressure such that it remains within a predetermined PEEP pressure range. To provide an example, for an infant or neonate, the targeted PEEP pressure range may be around 5 cm H2O, with a variation of −2 to +2 cm H2O. That is, the predetermined and acceptable PEEP pressure range may be around 3 to 7 cm H2O. In this case, the selected pressure level, at which point the controller 510 is lifted the valve seat 523, may be set at 3.5 to 4.5 cm H2O. This ensures that the valve 50 remains open during PEEP delivery, as long as the pressure is above the selected pressure level. The gas flow rate that the valve 50 is configured to regulate may be within a range of 0 to 20 L/min when used with infants or neonates. This range is approximately the same as the source flow rate which the respiratory system 1 is set to provide to a patient.

In FIG. 56B, the operator begins to press down the actuator 2801 (position 2—partially inserted position of the actuator 2801), until the actuator 2801 reaches its fully or substantially inserted position as indicated in FIG. 29C at position 3. This transitions the respiratory system 1 from PEEP to PIP as the vent 2800 is gradually closed when the actuator 2801 is pressed from its fully or substantially lifted position to the fully or substantially inserted position. There is a pressure differential across the upper and lower surfaces of the controller 510 until the actuator 2801 reaches its fully or substantially inserted position. In FIG. 56C, due to the sealing arrangement between the actuator 2801 and the vent housing 2803, the gas flow path within the pressure regulating device 70 is completely or substantially closed off. At this position, the gas within the respiratory system 1 is not allowed to flow through the pressure regulating device 70 anymore, causing PIP to be delivered to the patient.

In FIG. 56C, the controller 510 is biased against the valve seat 523, during delivery of PIP. When the vent 2800 is fully closed, the flow path within the pressure regulating device 70 is closed off, meaning gas will not flow past the controller 510 anymore, and the pressure differential across the controller becomes zero (i.e. the pressure applied to the upper and lower surfaces of the controller 510 may both equal to PIP pressure currently administered to the patient). As the pressure differential is zero, F_{lift} also equals zero. The forces that are exerted onto the controller 510, are the biasing force caused by the biasing member 530, and the equal and opposite force of the valve seat, which maintains the controller 510 in its seated position.

As the respiratory system 1 transitions from PIP to PEEP, the actuator 2801 is allowed to return to its lifted position, by reducing or removing the pressing force applied onto the actuator 2801. As the actuator 2801 returns to its fully or substantially lifted position, the vent 2800 is open to allow gases to exit to ambient air. This has an effect of introducing a differential pressure across the upper and lower surfaces of the controller 510, as the pressure applied to the upper surface of the controller 510 no longer equals PIP. If this pressure differential is greater than the pressure required to lift the controller 510 off the valve seat 523, the controller 510 is displaced away from the valve seat 523, as indicated in FIG. 56D, which opens up the valve 50. It will be understood that the drawings are for illustration purposes only and may not present every detail of the components and/or features. For example, for position 2 as illustrated in FIG. 56B and position 4 as illustrated in FIG. 56D, it will be understood that there is at least one path for gases to exit to the ambient air (or atmosphere) though such path(s) may not be clearly evident.

As mentioned previously, the vent of the present disclosure allows the respiratory system 1 to provide a relatively smooth transition when switching between different target PIP and PEEP pressures. That is, a range of intermediary pressures may also be provided to the patient for one or more time windows. An asymmetrical transition between PIP and PEEP may also be achieved with selected embodiments of the present disclosure. When the vent is used in conjunction with a flow independent PEEP valve, as described above, a greater certainty of PEEP pressures administered to the patient may be achieved. This is because the flow independent PEEP valve compensates for flow variations experienced near the patient's end, such that the PEEP pressure administered to the patient remains within a predetermined range, unaffected by the flow variations. This will give the operator a greater level of confidence that the desired respiratory therapy is supplied to the patient. In the event of auto-PEEP, the flow independent PEEP valve is able to displace the controller further away from its valve seat, to let more gases flow out of the respiratory system 1, such that auto-PEEP does not unintentionally raise the PEEP pressure to a higher level. In the event of unintentional leaks, for example, at the patient interface, the flow independent PEEP valve is able to automatically adjust the position of the controller again, such that the leaked gases have a reduced impact on the PEEP pressure, and the PEEP delivered to the patient still maintains within the predetermined PEEP pressure range.

FIGS. 57 to 61 illustrate further applications of a vent or a pressure regulating device as presently disclosed. In at least some of the previous embodiments described above, the vent or pressure regulating device may be attachable to or formed as part of a T-piece device. A releasable connection mechanism may be used to reduce set-up complexity and enable resuscitation therapy to be provided through a different interface, for example through a CPAP interface or other sealing interface.

FIGS. 57, 58, 60 and 61 illustrate examples of a pressure regulating device 70 including an example of the disclosed vent as releasably connected to a CPAP interface. Such attachment can enable a clinician to modify the CPAP interface to deliver resuscitation therapy, e.g. to administer resuscitation or rescue breaths to a patient as required. Such modification can take place whilst the CPAP interface remains on the patient.

FIGS. 59A to 59C show examples of releasable attachment mechanisms which may be used to transition between therapies using a single type of patient interface. In the embodiment shown in FIG. 59A, a connector portion 660 is provided to the pressure regulating device 70, allowing detachment/attachment of the regulating device 70 to other locations of the CPAP respiratory system 1a. As illustrated in FIG. 59A (see-through view of the connector portion 660 engaged with the pressure regulating device 70), the connector portion 660 is configured to engage with a male connector portion 661 to form a releasable connection. The male connector portion 661 may include one or more locking fingers. The or each locking finger may have a recess or aperture on an outer surface thereof. The recess or aperture may be configured to engage with a corresponding locking tab (not shown), located on an internal surface or wall of the connector portion 660. Similar releasable attachment mechanisms are described in international patent application number PCT/NZ2012/000142, the contents of which are incorporated herein in its entirety.

FIG. 59B (side view of the connector portion 660 engaged with the pressure regulating device 70) and 59C (side view of the connector portion 660 disengaged with the pressure regulating device 70) show a further example of a releasable attachment mechanism. In this example, connector portion 660 is a male connector portion, configured to engage with female connector portion of pressure regulating device 70 by friction or interference fit. Connector portion 660 may be configured to have a tapering profile, enabling direct insertion into the pressure regulating device 70. Alternatively, connector portion 660 may be a female connector portion, configured to engage with a male connector portion, e.g. provided to the pressure regulating device 70.

FIGS. 57 and 58 show examples of how the pressure regulating device 70 with releasable attachment mechanism may be used with an exemplary CPAP respiratory system 1a. In the illustrated arrangement, the patient interface 340 receives an inspiratory flow of gases via a first breathing conduit 210a. A flow of the expiratory gases can be directed from the interface 340 via a second breathing conduit 210b to the pressure regulating device 70. It may be appreciated that the pressure regulating device 70 may be replaced with another pressure regulating device or a vent of the present disclosure or a flow resistance device (e.g., a valve) to control the pressure delivered to the patient.

FIGS. 60 and 61 illustrate another example using a pressure regulating device or a vent of the present disclosure (e.g., the pressure regulating device 70) with a CPAP respiratory system 1a, in combination with a T-piece device (e.g., the device 320, 620, 1120). Similarly, the connector portion 660 is provided, allowing detachment/attachment of the T-piece device to an expiratory breathing conduit 210b of the CPAP respiratory system 1a. With reference to FIG. 61, the male connector portion 661 is provided to the expiratory conduit 201b. A corresponding female connector portion can be provided to an inlet (e.g., the inlet 324, 624, 1124) of the T-piece device. This configuration allows a quick connection to be made between the T-piece device and the CPAP interface 340. Alternatively, a female connector portion (not shown) at end of the expiratory conduit 201b can engage with a corresponding male connector portion e.g., provided to the inlet of the T-piece device.

The pressure regulating device 70, which can be coupled to a patient interface such as CPAP interface, can enable ready transition between different therapy types. For example, a patient receiving CPAP therapy may require additional respiratory support, such as resuscitation or rescue breath(s). A pressure regulating device 70 with releasable attachment mechanism can be connected to the CPAP interface, such as described above. The pressure regulating device 70 can be operated as described to deliver resuscitation therapy/rescue breaths. Upon the patient stabilising sufficiently, the patient can be transitioned back to CPAP therapy by disengaging the pressure regulating device 70 from expiratory conduit 210b and re-engaging expiratory resistance device, such as bubbler device (not shown).

Positive End Expiratory Pressure (PEEP) is also known as Peak End Expiratory Pressure and the two terms are often used interchangeably in the context of respiratory therapy systems and methods.

In this specification, adjectives such as left and right, top and bottom, hot and cold, first and second, and the like may be used to distinguish one element or action from another element or action without necessarily requiring or implying any actual such relationship or order. Where context permits, reference to a component, an integer or step (or the alike) is not to be construed as being limited to only one of that component, integer, or step, but rather could be one or more of that component, integer or step.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

The above description relating to embodiments of the present disclosure is provided for purposes of description to one of ordinary skill in the related art. It is not intended to be exhaustive or to limit the disclosure to a single disclosed embodiment. As mentioned above, numerous alternatives and variations to the present disclosure will be apparent to those skilled in the art from the above teaching. Accordingly, while some alternative embodiments have been discussed specifically, other embodiments will be apparent or relatively easily developed by those of ordinary skill in the art. The present disclosure is intended to embrace all modifications, alternatives, and variations that have been discussed herein, and other embodiments that fall within the spirit and scope of the above description.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

1.-73. (canceled)

74. A pressure regulating device for use with a respiratory system arranged to convey a breathable gas to a patient, the pressure regulating device comprising:
a vent, wherein the vent comprises:
a movable actuator, wherein movement of the actuator adjusts an area of the vent available for the gas to exit from the respiratory system via the pressure regulating device to regulate a pressure of the breathable gas within the respiratory system within a predetermined pressure range.

75. A respiratory system for delivering a respiratory therapy to a patient, the respiratory system comprising:
a respiratory apparatus, which supplies a source of breathable gas flow at a targeted pressure and/or flow rate;
a conduit assembly connectable to the respiratory apparatus to receive the breathable gas flow;
a patient interface, arranged to receive the breathable gas and usable to deliver the respiratory therapy to the patient;
a device arranged to form a fluid connection between the conduit assembly and the patient interface; and
a vent or a pressure regulating device including the vent,
wherein the vent comprises a movable actuator, and the pressure regulating device is configured to regulate a pressure of the breathable gas within the respiratory system within a predetermined pressure range, and wherein movement of the actuator adjusts an area of the vent available for the gas to exit from the respiratory system via the vent.

76. A kit of parts for use with a respiratory system, the kit of parts comprising:
a vent or a pressure regulating device including the vent,
wherein the vent comprises a movable actuator, and the pressure regulating device is configured to regulate a pressure of the breathable gas within the respiratory system within a predetermined pressure range, and wherein movement of the actuator adjusts an area of the vent available for the gas to exit from the respiratory system via the vent; and
a T-piece device, wherein the vent or the pressure regulating device is connectable to a PEEP port of the T-piece device.

77. (canceled)

78. A vent for use with a respiratory system arranged to convey a breathable gas to a patient, wherein the vent allows gas from within the respiratory system to exit, the vent comprising:
a movable actuator; and
a housing comprising a first opening fluidly connectable to the respiratory system, and a second opening adapted to receive the movable actuator,
wherein the actuator is arranged to move between a first position and a second position to adjust an area of the vent available for gas to exit from the respiratory system, thereby adjusting a pressure of the breathable gas delivered to the patient,
wherein in the first position, the actuator is lifted with respect to the housing, and in the second position, the actuator is inserted into the housing,
wherein when the actuator is in the first position, the pressure of the breathable gas delivered to the patient corresponds to positive end expiratory pressure (PEEP), and when the actuator is in the second position, the pressure of the breathable gas delivered to the patient corresponds to peak inspiratory pressure (PIP).

79. The vent of claim 78, wherein the actuator is arranged to be slid in or out of the housing via the second opening.

80. The vent of claim 78, wherein the actuator includes:
a body portion, including a first end and a tapering end, wherein a diameter of the body portion decreases towards the tapering end.

81. The vent of claim 78, wherein when the actuator is in the first position, the gas is allowed to flow via a gap between the actuator and an internal wall of the housing; and when the actuator is in the second position, the gap between the actuator and the internal wall of the housing is substantially blocked, such that the gas does not flow through the gap.

82. The vent of claim 78, wherein as the actuator is moved between the first and second positions, pressures between PEEP and PIP are delivered to the patient.

83. The vent of claim 78, wherein the actuator is manually operated by an operator to move between the first position and the second position.

84. The vent of claim 83, wherein the vent further includes a biasing member to cause the actuator to remain in the first position when no force is applied by the operator.

85. The vent of claim 78, wherein a sealing member is provided to create a seal when the actuator is in the first position.

86. The vent of claim 78, wherein the first opening has a diameter smaller than the second opening.

87. The vent of claim 78, wherein the housing comprises a body extending between the first and the second opening, wherein the body includes an orifice adapted to allow the gas to escape to atmosphere depending on a relative position of the actuator with respect to the housing.

88. The vent of claim 87, wherein the orifice includes a plurality of orifices.

89. The vent of claim 88, wherein the plurality of orifices are disposed around a circumference of the body and extend along a length of the body.

90. The vent of claim 78, wherein the actuator includes:
a body portion, including a first end and a tapering end, wherein a diameter of the body portion decreases towards the tapering end.

91. The vent of claim 78, wherein the actuator includes a substantially cylindrical body, wherein a bottom surface of the cylindrical body has a curved surface.

92. A pressure regulating device for use with a respiratory system arranged to convey a breathable gas to a patient, the pressure regulating device comprising:
an inlet arranged to receive a flow of breathable gas from a respiratory apparatus,
a first outlet connectable to a patient interface to deliver the breathable gas to the patient, and
a second outlet, wherein the second outlet is connected to a vent according to claim 78, and the second outlet is a PEEP port of the pressure regulating device.
